(19) United States
(12) Patent Application Publication
(10) Pub. No.: US 2008/0253976 A1
(43) Pub. Date: Oct. 16, 2008

(54) Personal Care Compositions Comprising An Antimicrobial Blend of Essential Oils or Constituents Thereof

(76) Inventors: Douglas Craig Scott, Loveland, OH (US); Lowell Alan Sanker, Cincinnati, OH (US); Eric Altman Goulbourne, Liberty Township, OH (US)

Correspondence Address: THE PROCTER & GAMBLE COMPANY;Global Legal Department - IP, Sycamore Building - 4th Floor, 299 East Sixth Street, CINCINNATI, OH, 45202, US

(21) Appl. No.: 12/062,870

(22) Filed: Apr. 4, 2008

Related U.S. Application Data
(60) Provisional application No. 60/923,652, filed on Apr. 16, 2007.

(51) Int. Cl. A61K 8/35; A61Q 11/00; A61P 31/00; A61Q 5/00; A61Q 19/00

(52) U.S. Cl. 424/49; 514/456; 424/70.1

(57) ABSTRACT

Disclosed are personal care compositions, including compositions for oral, throat and skin care comprising a blend of naturally occurring flavor or perfume ingredients or essential oils containing such ingredients, wherein the blend provides excellent antimicrobial activity and comprises at least two components, a first acyclic component selected from citral, neral, geranial, geraniol and nerol and a second cyclic-containing component selected from eucalyptol, carvacrol and eugenol. Preferably, the blend comprises 3, 4, 5 or more of the above components. Greater synergy in terms of antimicrobial efficacy may be obtained the more different components are blended together. The present compositions are effective in killing, suppressing the growth of and/or altering metabolism of microorganisms including those which cause undesirable oral cavity conditions including plaque, caries, calculus, gingivitis, periodontal disease and malodor. Optionally the blend further comprises additional antimicrobial and/or anti-inflammatory components, preferably naturally-occurring as well.

### CROSS REFERENCE TO RELATED APPLICATION

This application claims the benefit of U.S. Provisional Application Ser. No. 60/923, 652 filed on Apr. 16, 2007.

### TECHNICAL FIELD

The present invention relates to personal care compositions, such as products for oral, throat, nasal and skin care containing a blend of plant essential oils and/or their constituents to provide antimicrobial activity while providing a unique and pleasing flavor or scent that enhances consumer acceptability of the finished product. In particular for oral and throat care products, taste and mouthfeel characteristics are important not only for consumer acceptability but also to encourage compliance since use of these products may involve fairly long residence time in the mouth for efficacy.

### BACKGROUND OF THE INVENTION

Oral care products such as dentifrice and mouthrinse are routinely used by consumers as part of their oral care hygiene regimens to provide both therapeutic and cosmetic hygiene benefits. Therapeutic benefits include caries prevention which is typically delivered through the use of various fluoride salts; gingivitis prevention by the use of an antimicrobial agent such as triclosan, stannous fluoride, or essential oils; or hypersensitivity control through the use of ingredients such as stannous fluoride, strontium chloride or potassium nitrate. Hygiene and cosmetic benefits provided by oral care products include the control of plaque and calculus formation, removal and prevention of tooth stain, tooth whitening, breath freshening, and overall improvements in mouth feel impression which can be broadly characterized as mouth feel aesthetics. Calculus and plaque along with behavioral and environmental factors lead to formation of dental stains, significantly affecting the aesthetic appearance of teeth. Behavioral and environmental factors that contribute to teeth staining propensity include regular use of coffee, tea, cola or tobacco products, and also the use of certain oral products containing ingredients that promote staining, such as chlorhexidine and metal salts.

Dental plaque is a mixed matrix of bacteria, epithelial cells, leukocytes, macrophages and other oral exudates. Bacteria comprise approximately three-quarters of the plaque matrix. Any given sample of dental plaque could contain as many as 400 different varieties of microorganisms. This mix includes both aerobic and anaerobic bacteria, fungi, and protozoa. Viruses have also been found in samples of dental plaque. This matrix of organisms and oral exudates continues expanding and coalesces with other plaque growths situated nearby. The bacteria synthesize levans and glucans from sugars found in the oral cavity providing energy for the microorganisms. These glucans, levans, and microorganisms form an adhesive skeleton for the continued proliferation of plaque into what is also referred to as a biofilm, which is tenaciously adherent and difficult to remove. Mineralized dental plaque biofilms deposit on the surfaces of the teeth at the gingival margin and mature to what is referred to as calculus or tartar. As the mature calculus develops, it becomes visibly white or yellowish in color unless stained or discolored by some extraneous agent, becoming unsightly and undesirable from an aesthetic standpoint.

The failure to retard or stop the proliferation of plaque is detrimental to oral health, leading to dental caries, gingival inflammation, periodontal disease, and ultimately tooth loss. It is widely recognized that dental plaque bacteria, growing in the area where the teeth and gingival tissues meet, cause an inflammation of the gingiva called “gingivitis”. This is characterized by swollen, edematous gingiva (“gums”) which are reddened and bleed easily. If plaque removal is inadequate, gingivitis may progress to “periodontitis” or periodontal disease in many individuals. Periodontitis generally is characterized by a chronic inflammation of the tissues around the teeth, which leads to a resorption of supporting bone. Periodontal disease is the leading cause of tooth loss among adults. Dental caries (cavities) are also bacteria-mediated, with Streptococcus mutans believed to be the principal etiologic agent.

Prevention and removal of dental plaque have long been the focus of development, with the ultimate goal of inhibiting caries, calculus, gingivitis and periodontal diseases. While plaque removal can be accomplished to a certain extent by mechanical means such as by brushing the teeth particularly in conjuncton with abrasive compositions, brushing alone is not sufficient to effectively remove substantially all of the dental plaque that has formed on the teeth or prevent the formation or regrowth of plaque. To complement mechanical means of plaque control, chemical methods using antimicrobials have been proposed.

Among the many antimicrobial agents that have been demonstrated to be effective for use in the oral cavity include chlorhexidine; benzalkonium chloride; cetylpyridinium chloride; triclosan; metal ions such as stannous, zinc and copper; and essential oils. However, many of these oral antimicrobials have the disadvantage of causing negative aesthetics during use, in particular unpleasant taste and sensations and stain promotion. For example, chlorhexidine is one of the most effective antimicrobials but local side effects, notably unpleasant taste and staining limit its acceptability and long term use. In addition chlorohexidine and similar antibiotic actives such as doxycycline and metronidazole may have potential bacterial resistance issues along with a more widespread organism killing potential, i.e., both harmful and beneficial bacteria. For this reason and because consumers generally prefer products based on natural or naturally occurring ingredients as opposed to purely synthetic chemicals, there is an advantage in developing oral care products based on actives such as those derived from plant essential oils. Many of these essential oil actives are GRAS materials known to be safe for ingestion and effective to provide antimicrobial activity without harming beneficial oral microbial flora.

In one aspect, the present invention provides oral, nasal and throat care products comprising as antimicrobial active, a blend of selected materials that are naturally occurring in plant essential oils. In addition to the antimicrobial function, the present blend of particular essential oil ingredients provides a unique flavor base which can be combined with other typical flavoring agents such as mint oils, menthol, fruit oils, and coolants to provide pleasant tasting products that encourage user compliance with prescribed use.

In another aspect, antimicrobial topical compositions for use on skin, hair and other mucosal surfaces are provided utilizing the present blend of essential oil materials.

### SUMMARY OF THE INVENTION

The present invention is directed to personal care compositions, such as compositions for oral, throat and skin care comprising in a pharmaceutically acceptable carrier, a blend of naturally occurring flavor or perfume ingredients or essential oils containing such ingredients, wherein the blend exhibits excellent antimicrobial activity and comprises at least two components, a first component selected from acyclic structures including citral, neral, geranial, geraniol and nerol and a second component selected from cyclic or ring-containing structures including eucalyptol, eugenol and carvacrol. The present compositions are effective in killing, suppressing the growth of and/or altering metabolism of microorganisms such as those which cause undesirable conditions in the oral cavity including plaque, caries, calculus, gingivitis, periodontal disease and malodor. Optionally the blend further comprises other antimicrobially-effective and/or anti-inflammatory components, preferably naturally-occurring as well.

Oral, nasal and throat care products include products in powder, paste or liquid forms, which on being used are retained for a time sufficient to contact the surfaces and the internal mucous membranes of the oral or nasal cavities or the pharynx. Such products include for example, mouthwashes, dental and throat lozenges, gargles, chewing gum, dentifrice or toothpastes, throat sprays, toothpicks, dental tablets and powders and topical solutions for application in dental treatment, as well as cough syrups, chewable antacids and digestion promoting preparations. The present antimicrobial blend of naturally-occurring ingredients may also be incorporated in compositions for topical application to the skin, hair and other mucosal surfaces including lotions or creams, skin cleansers, shampoos and conditioners, cosmetic products such as lipsticks and foundations, wipes and towelettes and feminine hygiene products such as menstrual pads and tampons.

These and other features, aspects, and advantages of the present invention will become evident to those skilled in the art from the detailed description which follows.

### DETAILED DESCRIPTION OF THE INVENTION

While the specification concludes with claims particularly pointing out and distinctly claiming the invention, it is believed that the present invention will be better understood from the following description.

All percentages and ratios used hereinafter are by weight of total composition, unless otherwise indicated. All percentages, ratios, and levels of ingredients referred to herein are based on the actual amount of the ingredient, and do not include solvents, fillers, or other materials with which the ingredient may be combined as a commercially available product, unless otherwise indicated.

All measurements referred to herein are made at 25° C. unless otherwise specified.

Herein, “comprising” means that other steps and other components which do not affect the end result can be added. This term encompasses the terms “consisting of” and “consisting essentially of.”

As used herein, the word “include,” and its variants, are intended to be non-limiting, such that recitation of items in a list is not to the exclusion of other like items that may also be useful in the materials, compositions, devices, and methods of this invention.

As used herein, the words “preferred”, “preferably” and variants refer to embodiments of the invention that afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

By “oral care composition” is meant a product, which in the ordinary course of usage, is not intentionally swallowed for purposes of systemic administration of particular therapeutic agents, but is rather retained in the oral cavity for a time sufficient to contact substantially all of the dental surfaces and/or oral tissues for purposes of oral activity. The oral care composition may be in various forms including toothpaste, dentifrice, tooth gel, subgingival gel, mouthrinse, mousse, foam, denture product, mouthspray, lozenge, chewable tablet or chewing gum. The oral care composition may also be incorporated onto strips or films for direct application or attachment to oral surfaces.

The term “dentifrice”, as used herein, includes paste, gel, liquid, powder or tablet formulations unless otherwise specified. The dentifrice composition may be a single phase composition or may be a combination of two or more separate dentifrice compositions. The dentifrice composition may be in any desired form, such as deep striped, surface striped, multilayered, having a gel surrounding a paste, or any combination thereof. Each dentifrice composition in a dentifrice comprising two or more separate dentifrice compositions may be contained in a physically separated compartment of a dispenser and dispensed side-by-side.

The term “dispenser”, as used herein, means any pump, tube, or container suitable for dispensing compositions such as dentifrices.

The term “teeth”, as used herein, refers to natural teeth as well as artificial teeth or dental prosthesis.

The term “nasal and throat care composition” or “respiratory compositions” refer to compositions for use to treat respiratory conditions and which can be used herein in a form that is deliverable to a mammal in need. Nonlimiting examples include liquid compositions, nasal compositions, beverage, supplemental water, pills, soft gels, tablets, capsules, gel compositions, foam compositions, and combinations thereof. Nasal compositions, liquid compositions, gel compositions can be in a form that is directly deliverable to the nose, mouth and throat. These compositions and/or preparations can be delivered by a delivery device selected from droppers, pump, sprayers, liquid dropper, cup, bottle, liquid filled gel, liquid filled gummy, center filled gum, chews, films, center filled lozenge, gum filled lozenge, pressurized sprayers, atomizers, air inhalation devices, liquid filled compressed tablet, liquid filled gelatin capsule, liquid filled capsule, and other packaging and equipment, and combinations thereof. The sprayer, atomizer, and air inhalation devices can be associated with a battery or electric power source. For example, the respiratory compositions can be used to provide instant or on demand cough relief to a human.

The term “instant” and/or “on demand” as used herein refers to the compositions providing relief of one or more symptoms that is being treated, prevented, alleviated, ameliorated, inhibited, or mitigated within 20 minutes of application, alternatively within 15 minutes of application, alternatively within 10 minutes of application, alternatively within 5 minutes of application, alternatively within 2 minutes of application, alternatively within 1 minute of application.

The terms “pharmaceutically-acceptable carrier” or “orally-acceptable carrier” refer to safe and effective materials and conventional additives used in personal care compositions. For example, materials used in oral care compositions include but are not limited to one or more of fluoride ion sources, anti-calculus or anti-tartar agents, buffers, abrasives such as silica, alkali metal bicarbonate salts, thickening materials, humectants, water, surfactants, titanium dioxide, flavor system, sweetening agents, xylitol, and coloring agents.

The term “essential oils” as used herein refers to oils or extracts distilled or expressed from plants and constituents of these oils. Typical essential oils and their main constituents are those obtained for example from thyme (thymol, carvacrol), oregano (carvacrol, terpenes), lemon (limonene, terpinene, phellandrene, pinene, citral), lemongrass (citral, methylheptenone, citronellal, geraniol), orange flower (linalool, β-pinene, limonene), orange (limonene, citral), anise (anethole, safrol), clove (eugenol, eugenyl acetate, caryophyllene), rose (geraniol, citronellol), rosemary (borneol, bornyl esters, camphor), geranium (geraniol, citronellol, linalool), lavender (linalyl acetate, linalool), citronella (geraniol, citronellol, citronellal, camphene), eucalyptus (eucalyptol); peppermint (menthol, menthyl esters), spearmint (carvone, limonene, pinene); wintergreen (methyl salicylate), camphor (safrole, acetaldehyde, camphor), bay (eugenol, myrcene, chavicol), cinnamon (cinnamaldehyde, cinnamyl acetate, eugenol), tea tree (terpinen-4-ol, cineole), and cedar leaf (α-thujone, β-thujone, fenchone). Essential oils are widely used in perfumery and as flavorings, medicine and solvents. Essential oils, their composition and production, are described in detail in Kirk-Othmer Encyclopedia of Chemical Technology, 4^{th} Edition and in The Merck Index, 13^{th} Edition.

Active and other ingredients useful herein may be categorized or described by their cosmetic and/or therapeutic benefit or their postulated mode of action or function. However, it is to be understood that the active and other ingredients useful herein can, in some instances, provide more than one cosmetic and/or therapeutic benefit or function or operate via more than one mode of action. Therefore, classifications herein are made for the sake of convenience and are not intended to limit an ingredient to the particularly stated application or applications listed.

Herein, the terms “tartar” and “calculus” are used interchangeably and refer to mineralized dental plaque biofilms.

The essential and optional components of the present compositions are described in the following paragraphs.

In one embodiment of the present invention, oral care compositions are provided comprising a blend of naturally occurring flavor ingredients or essential oils (EO) containing such flavor ingredients, the blend exhibiting excellent antimicrobial activity and comprising at least two components, a first component selected from acyclic or non-ring structures including citral, neral, geranial, geraniol and nerol and a second component selected from ring-containing structures including eucalyptol,eugenol and carvacrol. Essential oils may be used to provide the above flavor ingredients including oils of lemongrass, citrus (orange, lemon, lime), citronella, geranium, rose, eucalyptus, oregano, bay and clove. However, it may be preferable that the flavor ingredients are provided as individual or purified chemicals rather than supplied in the composition by addition of natural oils or extracts as these sources may contain other components that may be unstable with other components of the composition or may introduce flavor notes that are incompatible with the desired flavor profile resulting in a less acceptable product from an organoleptic standpoint. Highly preferred for use herein are natural oils or extracts that have been purified or concentrated to contain mainly the desired component(s).

Preferably, the blend comprises 3, 4, 5 or more of the above components. Greater synergy in terms of antimicrobial efficacy may be obtained the more different components are blended together as long as the blend comprises at least one non-ring structure and one ring structure. A preferred blend comprises at least two ring structures or at least two non-ring structures. For example a blend comprising two non-ring structures (neral and geranial from citral) and eugenol as the ring structure is highly preferred for its efficacy against oral bacteria. Another preferred blend comprises three non-ring structures (geraniol, neral and geranial) and two ring structures (eugenol and eucalyptol).

Optionally the blend comprises additional antimicrobially-effective and/or anti-inflammatory components, preferably naturally-occurring as well. Such other antimicrobially-effective and/or anti-inflammatory components may include one or more of flavor/fragrance chemicals such as o-cymen-5-ol (isopropylmethylphenol, IPMP), farnesol, benzyl alcohol, benzaldehyde, hinokitiol (isopropyltropolone), terpinene-4-ol, zingerone, allyl isothiocyanate, cuminaldehyde, dipentene, α-pinene, β-pinene, menthol, methyl salicylate, anethole, carvone, limonene, ocimene, n-decyl alcohol, citronellal, citronellol, methyl acetate, citronellyl acetate, methyl eugenol, linalool, ethyl linalool, camphor, safrole, vanillin, chlorothymol, guaiacol, phenol, phenyl salicylate, cinnamaldehyde, cinnamic acid, guaiacol, isoeugenol, dihydroeugenol, vanillyl butyl ether, vanillin (4-formyl-guaiacol), 5-propenylguaethol, 4-ethyl-2-methoxyphenol, 4-allyl-2-methoxyphenol acetate, and 4-methyl guaiacol. Additional useful components having anti-inflammatory activity include flavonoids and flavones such as baicalein, baicalin, wogonoside, wogonin, and quercetin; phenolics such as catechin, gallocatechin gallate, epicatechin (EC), epigallocatechin (EGC), epigallocatechin gallate (EGCG), epicatechin gallate (ECG), theaflavine, thearubigins, anthocyanidins/proanthocyanidins and anthocyanins (e.g., cyanidin, delphinidin, pelargonidin, peonidin, malvidin and petunidin); tannic acid; gallic acid; ellagic acid; ellagitannins; hexamidine; and berberine. Natural sources of these chemicals may be used including oils, extracts or essences of spearmint, peppermint, wintergreen, lemon, orange, lime, cherry, sage, rosemary, cinnamon, cassia, oregano, ginger, basil, coriander, cilantro, allspice rose, tea tree (Melaleuca), pimento, laurel, anise, fennel, cumin, bay, bergamot, bitter almond, citronella, coal tar, lavender, mustard fennel, pine, pine needle, cedar leaf, sassafras, cubeb, spike lavender, creosote, horseradish, wasabi, tea, cranberry, pomegranate, oak bark and the like.

These flavor ingredients are among the hundreds of plant-sourced oils and extracts, constituents isolated therefrom and synthetic versions thereof that are commercially available. Many of these essential oils or individual flavor ingredients have been reported to have antimicrobial activity. However, the activity of individual components is typically too weak to be of practical use, unless combined with other antimicrobials or used at fairly high concentrations. The present inventors have found that a blend comprising at least one first component selected from citral neral, geranial, geraniol and nerol and at least one second component selected from eucalyptol, eugenol and carvacrol, provides effective antimicrobial action as well as an acceptable taste when incorporated into oral and throat care products such as dentifrice, mouthrinse and throat spray. It is important for oral and throat care products to have acceptable taste since these require fairly long residence time in the mouth for efficacy. The present blend provides formulation flexibility in that the amount of each component in the blend can be adjusted to derive maximum consumer appeal in terms of flavor and taste while providing the required antimicrobial efficacy. The present blend delivers the desired antimicrobial activity without requiring any particular component to be present in large quantities that may introduce flavor notes that may be incompatible with the overall flavor perception desired in the final product.

It is noteworthy that the present blend of essential oils does not include thymol to provide antimicrobial activity, although thymol may be included in the overall flavor system to add a certain “note”. Thymol is well-known for its antimicrobial activity and has been utilized in oral care preparations in sufficient quantities to provide beneficial therapeutic effects. For example, the combination of thymol with three other essential oils, menthol, eucalyptol and methyl salicylate is listed as the antiplaque/antigingivitis active ingredient in currently marketed mouthrinses under the Listerine® brand name. However, while thymol provides beneficial therapeutic effects, it also provides the consumer with a flavor perception that can be described as unpleasant, harsh or medicinal in taste. To this end, there have been attempts at masking the taste of thymol to improve consumer acceptability of the product such as described in U.S. Pat. No. 4,945,087 to Talwar, et al. Effective taste masking of thymol is reportedly accomplished by utilizing specified amounts of a sugar alcohol or a mixture of sugar alcohol and anethole.

The selection of the essential oils or components thereof to include in the present blend is based on demonstration of their activity against microorganisms known to be involved in undesirable oral cavity conditions such as gingivitis, periodontal disease and oral malodor, in particular bacteria such as P. gingivalis and F. nucleatum and other oral cavity strains including B. forsythus, A. actinomycetemcomitans, T. denticola, T. socranskii, P. intermedia, L. acidophilus, L. casei, A. viscosus, S. sobrinus, S sanguis, S. viridans, and S. mutans.

Periodontal disease may involve one or more of the following conditions: inflammation of the gingiva, formation of periodontal pockets, bleeding and/or pus discharge from the periodontal pockets, resorption of alveolar bone, loose teeth and loss of teeth. Bacteria present in dental plaque which forms on the surface of the teeth and in the periodontal pocket contribute to both the initiation and progress of periodontal disease. Thus, in order to prevent or treat periodontal disease, these bacteria must be suppressed by some means other than simple mechanical scrubbing. Towards this end, there has been a great deal of research aimed at developing therapeutic dentifrices, mouthwashes, and methods of treating periodontal disease, which are effective in suppressing these bacteria. However, periodontal disease involves more than just the bacterial infection. Severe periodontal disease involves the destruction of periodontal tissue, which is primarily caused by the indirect effects mediated by the host's reaction to the bacteria in the periodontium and gingival sulcus, specifically inflammation of the gingival and periodontium, or gingivitis. If left unchecked, gingivitis may progress into periodontitis, which may result in attachment loss, bone destruction and tooth loss. Anaerobic bacteria are generally regarded as the initiating agent of gingivitis, with subsequent progression and disease severity determined by the host immune response, i.e., inflammation, which is a nonspecific cellular and biochemical process involving multiple pro-inflammatory agents.

Bacterial metabolites induce leukocyte chemotaxis which results in the accumulation of inflammatory cells at the site of the bacterial challenge. Furthermore, bacterial metabolites induce the production of inflammatory mediators by leukocytic cells, in particular monocytes. Amongst these are local disease mediators such as metabolites of arachidonic acid, e.g., leukotrienes, prostaglandins and thromboxanes. Prostaglandins have been found to be particularly involved in the metabolism and destruction of tissue and alveolar bone. Indeed, the production of prostaglandins in the periodontal tissues has been found to be a key mediator of the loss of alveolar bone in the periodontium. Patients with periodontal breakdown show an elevated prostaglandin E₂ (PGE₂) level both in the gingival tissue as well as in the crevicular fluid. Prostaglandins and thromboxanes are formed from arachidonic acid by an enzyme cascade, the first step of which is the cyclooxygenation by an enzyme called cyclooxygenase (COX). Inhibiting the cyclooxygenase would inhibit the formation of prostaglandins and thus reduce alveolar bone loss. Indeed certain cyclooxygenase inhibitors, particularly non steroidal anti-inflammatory drugs such as indomethacin and flurbiprofen have been found to markedly reduce the resorption of alveolar bone.

Once inflammation starts, the process can self-propagate even when the causative agents, i.e., bacteria are removed. Therefore, an effective therapy for gingivitis would desirably include the combination of an antibacterial agent and an anti-inflammatory agent. Such combinations are disclosed for example in commonly assigned U.S. patent application Ser. No. 11/595,530, published as US 2007/0053849A1. The preferred actives disclosed therein are those having both antibacterial and anti-inflammatory activities.

The components of the present blend of essential oils exhibit both antibacterial and anti-inflammatory activities, and are thus particularly effective against bacteria-mediated inflammatory diseases such as gingivitis. The activities of the present essential oils are demonstrated using the assays described in the above cited application US 2007/0053849A1, including inhibitory activity against one or more of bacterial virulence and/or inflammation factors involving bacteria such as P. gingivalis.

P. gingivalis is a gram-negative anaerobe implicated in periodontal disease in humans and companion animals. P. gingivalis infects the gingival sulcus, producing a number of virulence factors including bacterial enzymes such as gingipains, METase and Cystalysin. Gingipains act on several immune system molecules, including kinogens, complement factors, immunoglobins, resulting in fluid influx into the sulcus, neutrophil recruitment, and bleeding. The ultimate result is a host inflammatory response characterized by cyclooxygenase (COX) induction, metalloproteinase (MMP) expression, prostaglandin elevation and reactive oxygen elevations which result in tissue damage and bone resorption, eventually leading to tooth detachment. MMP's (including various subtypes, e.g., MMP 1, 2, 3, 8, 9, 12, 13), are host extracellular matrix proteases that contribute to tissue destruction and remodeling. COX enzymes catalyze the conversion of arachidonic acid into prostaglandins, which result in vasodilation, redness, puffiness and pain.

Inhibition of the proteolytic action of gingipains on immuno-regulatory proteins should lead to a reduction in the inflammatory host-response and subsequent tissue damage. Other bacterial enzymes, for example, METase and Cystalysin, are involved in degrading sulfur-containing amino acids to produce volatile sulfur compounds (VSC's) such as hydrogen sulfide or methyl mercaptan that lead to bad breath or oral malodor. Inhibition of METase and Cystalysin is thus an important function for oral care agents as are inhibitory activities against key host pro-inflammatory factors including matrix metalloproteinases (MMP's), cyclooxygenases (COX) and prostaglandin (PGE₂).

Results of assays demonstrating activity of individual components of the present blend are summarized in Table 1 below. Where no data are reported is not an indication that the compound/active has no activity; rather that the compound was not tested for that activity.

TABLE 1
Inhibition of Bacterial Virulence Factors and Host Inflammation Factors

| Active | Gingipain | METase | Cystalysin | COX-1 | COX-2 | MMP (2, 3, 12) | PGE₂ |
|---|---|---|---|---|---|---|---|
| Geraniol | | + | + | +++ | ++ | + | ++ |
| Citral | + | + | | | | + | +++ |
| Eugenol | + | + | + | ++ | ++ | + | ++++ |
| Eucalyptol | + | + | + | | | + | ++++ |
| Carvacrol | + | + | + | +++ | ++ | + | ++++ |

+ % inhibition <25% at 200 uM for compounds, 0.001% for oils/extracts
++ % inhibition ≧25% at 200 uM for compounds, 0.001% for oils/extracts
+++ % inhibition ≧50% at 200 uM for compounds, 0.001% for oils/extracts
++++ % inhibition ≧75% at 200 uM for compounds, 0.001% for oils/extracts

The present essential oil blend and individual components have also been demonstrated to provide microbial or germ kill efficacy using an in vitro model designed to evaluate the ability of prospective antimicrobial agents to inhibit the growth of an oral anaerobic bacterial biofilm containing Fusobacterium nucleatum. The method involves forming a bacterial biofilm in a plastic uncoated 12-well tissue culture plate. The culture material is removed from the wells and the biofilm is treated with a test solution or appropriate control usually for 30 seconds. Subsequently, the biofilm is washed twice with sterile saline and fresh medium is placed on the biofilm which is then incubated at 37° C. overnight. The next day, the culture in each well is suspended and the optical density determined to assess biofilm growth. Results of evaluations of essential oils are summarized in Table 2, showing the lowest concentration of essential oils that completely inhibited the growth of the biofilm following a 30 second exposure, i.e., minimum inhibitory concentration (MIC). These results demonstrate the germ kill effectiveness of the present essential oil components, which is comparable to thymol and the synergistic effects of combinations of essential oils, with MIC values as much as 5 times lower than the individual components. For example, the combinations with 3 components (carvacrol+eucalyptol+geraniol) and (carvacrol+citral+geraniol) completely inhibited biofilm growth at concentrations as low as 250 ppm total oil concentration.

TABLE 2
Inhibition of Biofilm Growth

| | | | Total Oil |
|---|---|---|---|
| | | | Level |
| | Total Oil Level | | Inhibiting |
| Single | Inhibiting Biofilm | | Biofilm |
| Component | Growth | Combinations of Oils | Growth |
| Eugenol | 0.2% | Citral + Eugenol | 0.05% |
| Eucalyptol | 0.1% | Carvacrol + Eucalyptol | 0.05% |
| (Cineole) | | | |
| Geraniol | 0.1% | Eucalyptol + Eugenol | 0.05% |
| Carvacrol | 0.1% | Geraniol + Eugenol | 0.05% |
| Citral | 0.2% | Carvacrol + Eucalyptol + | 0.025% |
| | | Geraniol | |
| Thymol | 0.1% | Carvacrol + Citral + | 0.025% |
| | | Geraniol | |

In further studies, individual essential oil compounds and blends were evaluated for germ kill efficacy against oral anaerobes. For comparison, a cetyl pyridinium chloride solution (350 ppm) and a commercially available essential oil mouthrinse (Listerine® Cool Mint with ˜0.258% essential oil level) were tested.

Aqueous solutions (comprising 1% poloxamer 407 and 15% ethanol) were prepared with the essential oil load fixed at 0.15%. This is a typical active concentration used in mouthrinse, which is comparable to about a 1.5% concentration in dentifrice to deliver a typical dose. The test solutions were used neat in this experiment. The essential oil compounds and blends were tested using the Plaque Chip Assay (PCA) method, developed in the Procter & Gamble laboratories. The PCA method measures a combination of the following parameters: antibacterial efficacy (bacteria or germ kill), biofilm dispersion efficacy/chemical removal (chemical cleaning, e.g., detergency without germ kill), biofilm prevention and/or inhibition efficacy (prevention of biofilm growth) and active substantivity (cumulative efficacy).

The PCA method uses unmodified, whole saliva (obtained from multiple healthy human donors) to grow a biofilm containing relevant oral species on the surface of hydroxyapatite (HA) discs (the ‘chip’) mounted in the cap of plastic tubes (Nunc™ Cryovial). HA chips are subjected to five one-minute treatment and re-growth cycles, designed to mimic typical oral care product usage (dentifrice, rinse, gel) over a 2½ day period. Fresh saliva is replaced after each treatment, wherein the saliva used serves both as an inoculum of relevant oral bacteria, and as a nutrient source for the growth of the biofilm. Sixteen hours after the final treatment cycle, the biofilm remaining on the chip is removed, and the number of viable bacteria contained within it are measured by standard agar plate counting on ETSA (Enriched Trypticase Soy Agar) and ETSA-NV (Enriched Trypticase Soy Agar Supplemented with Nalidixic Acid and Vancomycin) media. The number of colony forming units per unit volume (cfu/ml) is measured for Total Facultative Anaerobes (TFA) and Gram Negative Anaerobes (GNA). Results are summarized in Table 3 below reported as Log Reduction in TFA and GNA colony forming units provided by test formulations vs. control, i.e., water or base aqueous solution (comprising 1% poloxamer 407 and 15% ethanol). Reductions in TFA or GNA bacterial counts vs. control are reasonably predictive of in vivo efficacy, in particular plaque removal efficacy and breath malodor reduction. In this testing, synergies were observed for example with the CIT+EUG and GER+EUC combinations at the 50:50 level of each component. However, the components do not necessarily need to be present at the same level. For example, even better germ kill is provided by the GER+EUC pair at a 65:35 ratio compared to a 50:50 ratio. Particularly for blends containing 3 or more components, the relative amounts of each component can be adjusted to provide the best germ kill efficacy along with the desired flavor profile.

TABLE 3
Log Reduction in TFA and GNA Counts

| | Log Reduction | |
|---|---|---|
| | in cfu/ml vs | |
| | Control* | |
| Essential Oil at 0.15% Solution | TFA | GNA |
| Carvacrol (CAR) | 0.08 | 0.42 |
| Eucalyptol (EUC) | −0.06 | 0.38 |
| Eugenol (EUG) | 0.09 | 0.70 |
| Citral (CIT) | 0.33 | 0.72 |
| Geraniol (GER) | 0.47 | 0.60 |
| 50% CAR + 50% EUC | 0.02 | 0.60 |
| 50% CAR + 50% EUG | 0.02 | 0.52 |
| 50% CAR + 50% CIT | 0.22 | 0.37 |
| 50% CAR + 50% GER | 0.35 | 0.66 |
| 50% EUC + 50% EUG | 0.01 | 0.51 |
| 50% EUC + 50% CIT | 0.34 | 0.48 |
| 50% EUC + 50% GER | 0.84 | 0.70 |
| 50% EUG + 50% CIT | 1.17 | 1.11 |
| 50% EUG + 50% GER | 0.13 | 0.65 |
| 50% CIT + 50% GER | 0.51 | 0.61 |
| 20% each CAR + EUC + EUG + CIT + | 0.10 | 0.55 |
| GER | | |
| 12.5% CAR + 25% EUC + 25% EUG + 12.5% CIT + | 0.13 | 0.90 |
| 25% GER | | |
| 19.6% each of CAR, EUC, CIT, GER, IPMP + 2% EUG | 0.71* | 1.29* |
| 24.4% each of CAR, EUC, CIT, GER + 2.4% EUG | 0.58* | 1.07* |
| 350 ppm Cetylpyridinium Chloride (CPC) Soln. | 1.45 | 1.31 |
| Listerine ® Cool Mint (~0.258% EO level) | 0.14* | 0.45* |

*Values are reported vs. water/poloxamer/ethanol vehicle; all other values are reported vs. water.

The present antimicrobial blend comprising at least two, preferably three, more preferably four or more components selected from citral, neral, geranial, geraniol, nerol, carvacrol, eucalyptol and eugenol, is used at levels of at least about 0.02%, typically from about 0.05% to about 5.00% in the finished oral care product. In certain embodiments, the blend is present at levels of from about 0.05% to about 2.0%, from about 0.1% to about 1.5%, from about 0.3% to about 1.0%, or from about 0.5% to about 0.8% by weight of the composition.

The antimicrobial blend comprises at least about 0.5% by weight of each component, preferably at least about 1%, even more preferably at least about 5%, most preferably at least about 10%. In two component blends, the ratio of the first component to the second component will typically range from 20:80 to 80:20. For example, a two component blend may contain geraniol and eucalyptol at a 65:35 ratio. Another may contain citral and eugenol at a 50:50 ratio, although technically this blend contains three components since citral (from natural sources) is a mixture of the geometric isomers geranial and neral at about a 2:1 ratio. A four component blend may contain eugenol, eucalyptol, geranial and neral. Again citral may be used to provide geranial and neral. A five component blend may add geraniol to the four component blend above. In yet another embodiment the blend comprises six components, for example, from about 1.5% to about 20% citral (providing neral and geranial); from about 10 to about 50% geraniol; from about 10% to about 40% eucalyptol; from about 2% to about 25% eugenol and from about 2% to about 20-% carvacrol.

In other embodiments, the blend further comprises one or more different antimicrobially-effective and/or anti-inflammatory components in addition to or in place of one or more of the above essential oil actives. Preferably the other antimicrobially-effective component is selected from o-cymen-5-ol (isopropylmethylphenol or IPMP), menthol, carvone, cinnamaldehyde, anethole, terpinene-4-ol, zingerone, allyl isothiocyanate, cuminaldehyde, hinokitiol, α-pinene, β-pinene, dipentene, benzyl alcohol, benzaldehyde, guaiacol or natural sources thereof including oils or extracts of peppermint, spearmint, cinnamon, anise, fennel, tea tree, ginger, horseradish, wasabi, cumin, pine, cedar leaf, cubeb, cherry, creosote and the like.

The composition may optionally include additional ingredients such as mint-type oils (spearmint, peppermint, wintergreen), fruit oils, spice oils, coolants and sweeteners as part of the flavor system.

Suitable cooling agents or coolants include a wide variety of materials such as menthol and derivatives thereof. Among synthetic coolants, many are derivatives of or are structurally related to menthol, i.e., containing the cyclohexane moiety, and derivatized with functional groups including carboxamide, ketal, ester, ether and alcohol. Examples include the ρ-menthanecarboxamide compounds such as N-ethyl-p-menthan-3-carboxamide, known commercially as “WS-3”, and others in the series such as WS-5, WS-11, WS-14 and WS-30. An example of a synthetic carboxamide coolant that is structurally unrelated to menthol is N,2,3-trimethyl-2-isopropylbutanamide, known as “WS-23”. Additional suitable coolants include 3-1-menthoxypropane-1,2-diol known as TK-10, isopulegol (under the tradename Coolact P) and ρ-menthane-3,8-diol (under the tradename Coolact 38D) all available from Takasago; menthone glycerol acetal known as MGA; menthyl esthers such as menthyl acetate, menthyl acetoacetate, menthyl lactate known as Frescolat® supplied by Haarmann and Reimer, and monomenthyl succinate under the tradename Physcool from V. Mane. The terms menthol and menthyl as used herein include dextro- and levorotatory isomers of these compounds and racemic mixtures thereof TK-10 is described in U.S. Pat. No. 4,459,425, Amano et al. WS-3 and other carboxamide cooling agents are described for example in U.S. Pat. Nos. 4,136,163; 4,150,052; 4,153,679; 4,157,384; 4,178,459 and 4,230,688. Additional N-substituted ρ-menthane carboxamides are described in WO 2005/049553A1 including N-(4-cyanomethylphenyl)-ρ-menthanecarboxamide, N-(4-sulfamoylphenyl)-ρ-menthanecarboxamide, N-(4-cyanophenyl)-ρ-menthanecarboxamide, N-(4-acetylphenyl)-ρ-menthanecarboxamide, N-(4-hydroxymethylphenyl)-ρ-menthanecarboxamide and N-(3-hydroxy-4-methoxyphenyl)-ρ-menthanecarboxamide.

Suitable sweeteners include those well known in the art, including both natural and artificial sweeteners. Some suitable water-soluble sweeteners include monosaccharides, disaccharides, polysaccharides and derivatives such as xylose, ribose, glucose (dextrose), mannose, galactose, fructose (levulose), sucrose (sugar), maltose, invert sugar (a mixture of fructose and glucose derived from sucrose), partially hydrolyzed starch, corn syrup solids, dihydrochalcones, monellin, steviosides, glycyrrhizin, xylitol and erythritol. Suitable water-soluble artificial sweeteners include soluble saccharin salts, i.e., sodium or calcium saccharin salts, cyclamate salts, the sodium, ammonium or calcium salt of 3,4-dihydro-6-methyl-1,2,3-oxathiazine-4-one-2,2-dioxide, the potassium salt of 3,4-dihydro-6-methyl-1,2,3-oxathiazine-4-one-2,2-dioxide (acesulfame-K), the free acid form of saccharin, and the like. Other suitable sweeteners include Dipeptide based sweeteners, such as L-aspartic acid derived sweeteners, such as L-aspartyl-L-phenylalanine methyl ester (aspartame) and materials described in U.S. Pat. No. 3,492,131, L-alpha-aspartyl-N-(2,2,4,4-tetramethyl-3-thietanyl)-D-alaninamide hydrate, methyl esters of L-aspartyl-L-phenylglycerin and L-aspartyl-L-2,5,dihydrophenyl-glycine, L-aspartyl-2,5-dihydro-L-phenylalanine, L-aspartyl-L-(1-cyclohexyen)-alanine, and the like. Water-soluble sweeteners derived from naturally occurring water-soluble sweeteners, such as a chlorinated derivative of ordinary sugar (sucrose), known, for example, under the product description of sucralose as well as protein based sweeteners such as thaumatoccous danielli (Thaumatin I and II) can be used. A composition preferably contains from about 0.1% to about 10% of sweetener, preferably from about 0.1% to about 1%, by weight of the composition.

The flavor system may also include salivating agents, warming agents, and numbing agents. These agents are present in the compositions at a level of from about 0.001% to about 10%, preferably from about 0.1% to about 1%, by weight of the composition. Suitable salivating agents include Jambu® manufactured by Takasago and Optaflow® from Symrise .Examples of warming agents are capsicum and nicotinate esters, such as benzyl nicotinate. Suitable numbing agents include benzocaine, lidocaine, clove bud oil, and ethanol.

In addition to the components described above, the present compositions may comprise additional optional components collectively referred to as orally acceptable carrier materials, which are described in the following paragraphs.

### Orally Acceptable Carrier Materials

Orally acceptable carrier materials include one or more compatible solid or liquid excipients or diluents which are suitable for topical oral administration. By “compatible” is meant that the components of the composition are capable of being commingled without interaction in a manner which would substantially reduce composition stability and/or efficacy.

The carriers or excipients of the present invention can include the usual and conventional components of dentifrices, non-abrasive gels, subgingival gels, mouthwashes or rinses, mouth sprays, chewing gums, lozenges and breath mints as more fully described hereinafter.

The choice of a carrier to be used is basically determined by the way the composition is to be introduced into the oral cavity. Carrier materials for toothpaste, tooth gel or the like include abrasive materials, sudsing agents, binders, humectants, flavoring and sweetening agents, etc. as disclosed in e.g., U.S. Pat. No. 3,988,433 to Benedict. Carrier materials for biphasic dentifrice formulations are disclosed in U.S. Pat. Nos. 5,213,790; 5,145,666 and 5,281,410 all to Lukacovic et al. and in U. S. Pat. Nos. 4,849,213 and 4,528,180 to Schaeffer. Mouthwash, rinse or mouth spray carrier materials typically include water, flavoring and sweetening agents, etc., as disclosed in, e.g., U.S. Pat. No. 3,988,433 to Benedict. Lozenge carrier materials typically include a candy base; chewing gum carrier materials include a gum base, flavoring and sweetening agents, as in, e.g., U.S. Pat. No. 4,083,955 to Grabenstetter et al. Sachet carrier materials typically include a sachet bag, flavoring and sweetening agents. For subgingival gels used for delivery of actives into the periodontal pockets or around the periodontal pockets, a “subgingival gel carrier” is chosen as disclosed in, e.g. U.S. Pat. Nos. 5,198,220 and 5,242,910 both to Damani. Carriers suitable for the preparation of compositions of the present invention are well known in the art. Their selection will depend on secondary considerations like taste, cost, and shelf stability, etc.

The compositions of the present invention may also be in the form of non-abrasive gels and subgingival gels, which may be aqueous or non-aqueous. In still another aspect, the invention provides a dental implement impregnated with the present composition. The dental implement comprises an implement for contact with teeth and other tissues in the oral cavity, said implement being impregnated with the present composition. The dental implement can be impregnated fibers including dental floss or tape, chips, strips, films and polymer fibers.

In one embodiment, the compositions of the subject invention are in the form of dentifrices, such as toothpastes, tooth gels, tooth powders and tablets. Components of such toothpaste and tooth gels generally include one or more of a dental abrasive (from about 6% to about 50% ), a surfactant (from about 0.5% to about 10% ), a thickening agent (from about 0.1% to about 5%), a humectant (from about 10% to about 55%), a flavoring agent (from about 0.04% to about 2%), a sweetening agent (from about 0.1% to about 3%), a coloring agent (from about 0.01% to about 0.5%) and water (from about 2% to about 45%). Such toothpaste or tooth gel may also include one or more of an anticaries agent (from about 0.05% to about 0.3% as fluoride ion) and an anticalculus agent (from about 0.1% to about 13%). Tooth powders, of course, contain substantially all non-liquid components.

Other embodiments of the subject invention are liquid products, including mouthwashes or rinses, mouth sprays, dental solutions and irrigation fluids. Components of such mouthwashes and mouth sprays typically include one or more of water (from about 45% to about 95%), ethanol (from about 0% to about 25%), a humectant (from about 0% to about 50%), a surfactant (from about 0.01% to about 7%), a flavoring agent (from about 0.04% to about 2%), a sweetening agent (from about 0.1% to about 3%), and a coloring agent (from about 0.001% to about 0.5%). Such mouthwashes and mouth sprays may also include one or more of an anticaries agent (from about 0.05% to about 0.3% as fluoride ion) and an anticalculus agent (from about 0.1% to about 3%). Components of dental solutions generally include one or more of water (from about 90% to about 99%), preservative (from about 0.01% to about 0.5%), thickening agent (from 0% to about 5%), flavoring agent (from about 0.04% to about 2%), sweetening agent (from about 0.1% to about 3%), and surfactant (from 0% to about 5%).

Types of orally acceptable carriers or excipients which may be included in compositions of the present invention, along with specific non-limiting examples, are discussed in the following paragraphs.

### Other Active Agents

The present compositions may optionally include other agents, such as other antimicrobial agents. Included among such agents are water insoluble non-cationic antimicrobial agents such as halogenated diphenyl ethers, phenolic compounds including phenol and its homologs, mono and poly-alkyl and aromatic halophenols, resorcinol and its derivatives, bisphenolic compounds and halogenated salicylanilides, benzoic esters, and halogenated carbanilides. The water soluble antimicrobials include quaternary ammonium salts and bis-biquanide salts, and triclosan monophosphate. The quaternary ammonium agents include those in which one or two of the substituents on the quaternary nitrogen has a carbon chain length (typically alkyl group) from about 8 to about 20, typically from about 10 to about 18 carbon atoms while the remaining substitutents (typically alkyl or benzyl group) have a lower number of carbon atoms, such as from about 1 to about 7 carbon atoms, typically methyl or ethyl groups. Dodecyl trimethyl ammonium bromide, tetradecylpyridinium chloride, domiphen bromide, N-tetradecyl-4-ethyl pyridinium chloride, dodecyl dimethyl (2-phenoxyethyl) ammonium bromide, benzyl dimethylstearyl ammonium chloride, cetyl pyridinium chloride, quatemized 5-amino-1,3-bis(2-ethyl-hexyl)-5-methyl hexa hydropyrimidine, benzalkonium chloride, benzethonium chloride and methyl benzethonium chloride are exemplary of typical quaternary ammonium antibacterial agents. Other compounds are bis[4-(R-amino)-1-pyridinium] alkanes as disclosed in U.S. Pat. No. 4,206,215, issued to Bailey. Other antimicrobials such as copper salts, zinc salts and stannous salts may also be included. Also useful are enzymes, including endoglycosidase, papain, dextranase, mutanase, and mixtures thereof. Such agents are disclosed in U.S. Pat. No. 2,946,725 to Norris et al. and in U.S. Pat. No. 4,051,234 to Gieske et al. Preferred antimicrobial agents include zinc salts, stannous salts, cetyl pyridinium chloride, chlorhexidine, triclosan, triclosan monophosphate, and flavor oils. Triclosan and other agents of this type are disclosed in Parran, Jr. et al., U.S. Pat. No. 5,015,466 and U.S. Pat. No. 4,894,220 to Nabi et al. These agents provide anti-plaque benefits and are typically present at levels of from about 0.01% to about 5.0%, by weight of the composition.

Another optional active agent that may be added to the present compositions is a dentinal desensitizing agent to control hypersensitivity, such as salts of potassium, calcium, strontium and tin including nitrate, chloride, fluoride, phosphates, pyrophosphate, polyphosphate, citrate, oxalate and sulfate.

### Anticalculus Agent

The present compositions may optionally include an anticalculus agent, such as a pyrophosphate salt as a source of pyrophosphate ion. The pyrophosphate salts useful in the present compositions include the dialkali metal pyrophosphate salts, tetraalkali metal pyrophosphate salts, and mixtures thereof. Disodium dihydrogen pyrophosphate (Na₂H₂P₂O₇), tetrasodium pyrophosphate (Na₄P₂O₇), and tetrapotassium pyrophosphate (K₄P₂O₇) in their unhydrated as well as hydrated forms are the preferred species. In compositions of the present invention, the pyrophosphate salt may be present in one of three ways: predominately dissolved, predominately undissolved, or a mixture of dissolved and undissolved pyrophosphate.

Compositions comprising predominately dissolved pyrophosphate refer to compositions where at least one pyrophosphate ion source is in an amount sufficient to provide at least about 1.0% free pyrophosphate ions. The amount of free pyrophosphate ions may be from about 1% to about 15%, from about 1.5% to about 10% in one embodiment, and from about 2% to about 6% in another embodiment. Free pyrophosphate ions may be present in a variety of protonated states depending on the pH of the composition.

Compositions comprising predominately undissolved pyrophosphate refer to compositions containing no more than about 20% of the total pyrophosphate salt dissolved in the composition, preferably less than about 10% of the total pyrophosphate dissolved in the composition. Tetrasodium pyrophosphate salt is a preferred pyrophosphate salt in these compositions. Tetrasodium pyrophosphate may be the anhydrous salt form or the decahydrate form, or any other species stable in solid form in the dentifrice compositions. The salt is in its solid particle form, which may be its crystalline and/or amorphous state, with the particle size of the salt preferably being small enough to be aesthetically acceptable and readily soluble during use. The amount of pyrophosphate salt useful in making these compositions is any tartar control effective amount, generally from about 1.5% to about 15%, preferably from about 2% to about 10%, and most preferably from about 3% to about 8%, by weight of the dentifrice composition.

Compositions may also comprise a mixture of dissolved and undissolved pyrophosphate salts. Any of the above mentioned pyrophosphate salts may be used.

The pyrophosphate salts are described in more detail in Kirk-Othmer Encyclopedia of Chemical Technology, Third Edition, Volume 17, Wiley-Interscience Publishers (1982).

Optional agents to be used in place of or in combination with the pyrophosphate salt include such known materials as synthetic anionic polymers, including polyacrylates and copolymers of maleic anhydride or acid and methyl vinyl ether (e.g., Gantrez), as described, for example, in U.S. Pat. No. 4,627,977, to Gaffar et al., as well as, e.g., polyamino propane sulfonic acid (AMPS), diphosphonates (e.g., EHDP; AHP), polypeptides (such as polyaspartic and polyglutamic acids), and mixtures thereof.

### Fluoride Source

It is common to have a fluoride compound present in dentifrices and other oral compositions in an amount sufficient to give a fluoride ion concentration in the composition, and/or when it is used of from about 0.0025% to about 5.0% by weight, preferably from about 0.005% to about 2.0% by weight, to provide anticaries effectiveness. A wide variety of fluoride ion-yielding materials can be employed as sources of soluble fluoride in the present compositions. Examples of suitable fluoride ion-yielding materials are found in U.S. Pat. No. 3,535,421 to Briner et al. and U.S. Pat. No. 3,678,154 to Widder et al. Representative fluoride ion sources include: stannous fluoride, sodium fluoride, potassium fluoride, sodium monofluorophosphate, indium fluoride and many others.

### Abrasives

Dental abrasives useful in the compositions of the subject invention include many different materials. The material selected must be one which is compatible within the composition of interest and does not excessively abrade dentin. Suitable abrasives include, for example, silicas including gels and precipitates, insoluble sodium polymetaphosphate, hydrated alumina, calcium carbonate, dicalcium orthophosphate dihydrate, calcium pyrophosphate, tricalcium phosphate, calcium polymetaphosphate, and resinous abrasive materials such as particulate condensation products of urea and formaldehyde.

Another class of abrasives for use in the present compositions is the particulate thermo-setting polymerized resins as described in U.S. Pat. No. 3,070,510 issued to Cooley & Grabenstetter. Suitable resins include, for example, melamines, phenolics, ureas, melamine-ureas, melamine-formaldehydes, urea-formaldehyde, melamine-urea-formaldehydes, cross-linked epoxides, and cross-linked polyesters.

Silica dental abrasives of various types are preferred because of their unique benefits of exceptional dental cleaning and polishing performance without unduly abrading tooth enamel or dentine. The silica abrasive polishing materials herein, as well as other abrasives, generally have an average particle size ranging between about 0.1 to about 30 microns, and preferably from about 5 to about 15 microns. The abrasive can be precipitated silica or silica gels such as the silica xerogels described in Pader et al., U.S. Pat. No. 3,538,230 and DiGiulio, U.S. Pat. No. 3,862,307. Examples include the silica xerogels marketed under the trade name “Syloid” by the W.R. Grace & Company, Davison Chemical Division and precipitated silica materials such as those marketed by the J. M. Huber Corporation under the trade name, Zeodent®, particularly the silicas carrying the designation Zeodent® 119, Zeodent® 118, Zeodent® 109 and Zeodent® 129. The types of silica dental abrasives useful in the toothpastes of the present invention are described in more detail in Wason, U.S. Pat. No. 4,340,583; and in commonly-assigned U.S. Pat. Nos. 5,603,920; 5,589,160; 5,658,553; 5,651,958; and 6,740,311.

Mixtures of abrasives can be used such as mixtures of the various grades of Zeodent® silica abrasives listed above. The total amount of abrasive in dentifrice compositions of the subject invention typically range from about 6% to about 70% by weight; toothpastes preferably contain from about 10% to about 50% of abrasives. Dental solution, mouth spray, mouthwash and non-abrasive gel compositions of the subject invention typically contain little or no abrasive.

### Tooth Substantive Agent

The present invention may include a tooth substantive agent such as polymeric surface active agents (PMSA's), which are polyelectrolytes, more specifically anionic polymers. The PMSA's contain anionic groups, e.g., phosphate, phosphonate, carboxy, or mixtures thereof, and thus, have the capability to interact with cationic or positively charged entities. The “mineral” descriptor is intended to convey that the surface activity or substantivity of the polymer is toward mineral surfaces such as calcium phosphate minerals or teeth.

PMSA's are useful in the present compositions because of their stain prevention benefit. It is believed the PMSA's provide a stain prevention benefit because of their reactivity or substantivity to mineral surfaces, resulting in desorption of portions of undesirable adsorbed pellicle proteins, in particular those associated with binding color bodies that stain teeth, calculus development and attraction of undesirable microbial species. The retention of these PMSA's on teeth can also prevent stains from accruing due to disruption of binding sites of color bodies on tooth surfaces.

The ability of PMSA's to bind stain promoting ingredients of oral care products, for example, stannous ions and cationic antimicrobials, is also believed to be helpful. The PMSA will also provide tooth surface conditioning effects which produce desirable effects on surface thermodynamic properties and surface film properties, which impart improved clean feel aesthetics both during and most importantly, following rinsing or brushing. Many of these polymeric agents are also known or expected to provide tartar control benefits when applied in oral compositions, hence providing improvement in both the appearance of teeth and their tactile impression to consumers.

The desired surface effects include: 1) creating a hydrophilic tooth surface immediately after treatment; and 2) maintaining surface conditioning effects and control of pellicle film for extended periods following product use, including post brushing or rinsing and throughout more extended periods. The effect of creating an increased hydrophilic surface can be measured in terms of a relative decrease in water contact angles. The hydrophilic surface, importantly, is maintained on the tooth surface for an extended period after using the product.

The polymeric mineral surface active agents include any agent which will have a strong affinity for the tooth surface, deposit a polymer layer or coating on the tooth surface and produce the desired surface modification effects. Suitable examples of such polymers are polyelectrolytes such as condensed phosphorylated polymers; polyphosphonates; copolymers of phosphate- or phosphonate-containing monomers or polymers with other monomers such as ethylenically unsaturated monomers and amino acids or with other polymers such as proteins, polypeptides, polysaccharides, poly(acrylate), poly(acrylamide), poly(methacrylate), poly(ethacrylate), poly(hydroxyalkylmethacrylate), poly(vinyl alcohol), poly(maleic anhydride), poly(maleate) poly(amide), poly(ethylene amine), poly(ethylene glycol), poly(propylene glycol), poly(vinyl acetate) and poly(vinyl benzyl chloride); polycarboxylates and carboxy-substituted polymers; and mixtures thereof Suitable polymeric mineral surface active agents include the carboxy-substituted alcohol polymers described in U.S. Pat. Nos. 5,292,501; 5,213,789, 5,093,170; 5,009,882; and 4,939,284; all to Degenhardt et al. and the diphosphonate-derivatized polymers in U.S. Pat. No. 5,011,913 to Benedict et al; the synthetic anionic polymers including polyacrylates and copolymers of maleic anhydride or acid and methyl vinyl ether (e.g., Gantrez), as described, for example, in U.S. Pat. No. 4,627,977, to Gaffar et al. A preferred polymer is diphosphonate modified polyacrylic acid. Polymers with activity must have sufficient surface binding propensity to desorb pellicle proteins and remain affixed to enamel surfaces. For tooth surfaces, polymers with end or side chain phosphate or phosphonate functions are preferred although other polymers with mineral binding activity may prove effective depending upon adsorption affinity.

Additional examples of suitable phosphonate containing polymeric mineral surface active agents include the geminal diphosphonate polymers disclosed as anticalculus agents in U.S. Pat. No. 4,877,603 to Degenhardt et al; phosphonate group containing copolymers disclosed in U.S. Pat. No. 4,749,758 to Dursch et al. and in GB 1,290,724 (both assigned to Hoechst) suitable for use in detergent and cleaning compositions; and the copolymers and cotelomers disclosed as useful for applications including scale and corrosion inhibition, coatings, cements and ion-exchange resins in U.S. Pat. No. 5,980,776 to Zakikhani et al. and U.S. Pat. No. 6,071,434 to Davis et al. Additional polymers include the water-soluble copolymers of vinylphosphonic acid and acrylic acid and salts thereof disclosed in GB 1,290,724 wherein the copolymers contain from about 10% to about 90% by weight vinylphosphonic acid and from about 90% to about 10% by weight acrylic acid, more particularly wherein the copolymers have a weight ratio of vinylphosphonic acid to acrylic acid of 70% vinylphosphonic acid to 30% acrylic acid; 50% vinylphosphonic acid to 50% acrylic acid; or 30% vinylphosphonic acid to 70% acrylic acid. Other suitable polymers include the water soluble polymers disclosed by Zakikhani and Davis prepared by copolymerizing diphosphonate or polyphosphonate monomers having one or more unsaturated C═C bonds (e.g., vinylidene-1,1-diphosphonic acid and 2-(hydroxyphosphinyl)ethylidene-1,1-diphosphonic acid), with at least one further compound having unsaturated C═C bonds (e.g., acrylate and methacrylate monomers). Suitable polymers include the diphosphonate/acrylate polymers supplied by Rhodia under the designation ITC 1087 (Average MW 3000-60,000) and Polymer 1154 (Average MW 6000-55,000).

A preferred PMSA will be stable with other components of the oral care composition such as ionic fluoride and metal ions. Also preferred are polymers that have limited hydrolysis in high water content formulations, thus permitting a simple single phase dentifrice or mouthrinse formulation. If the PMSA does not have these stability properties, one option is a dual phase formulation with the polymeric mineral surface active agent separated from the fluoride or other incompatible component. Another option is to formulate non-aqueous, essentially non-aqueous or limited water compositions to minimize reaction between the PMSA and other components.

A preferred PMSA is a polyphosphate. A polyphosphate is generally understood to consist of two or more phosphate molecules arranged primarily in a linear configuration, although some cyclic derivatives may be present. Although pyrophosphates (n=2) are technically polyphosphates, the polyphosphates desired are those having around three or more phosphate groups so that surface adsorption at effective concentrations produces sufficient non-bound phosphate functions, which enhance the anionic surface charge as well as hydrophilic character of the surfaces. The inorganic polyphosphate salts desired include tripolyphosphate, tetrapolyphosphate and hexametaphosphate, among others. Polyphosphates larger than tetrapolyphosphate usually occur as amorphous glassy materials. Preferred in this invention are the linear polyphosphates having the formula:

XO(XPO₃)ₙX

wherein X is sodium, potassium or ammonium and n averages from about 3 to about 125. Preferred polyphosphates are those having n averaging from about 6 to about 21, such as those commercially known as Sodaphos (n≈6), Hexaphos (n≈13), and Glass H (n≈21) and manufactured by FMC Corporation and Astaris. These polyphosphates may be used alone or in combination. Polyphosphates are susceptible to hydrolysis in high water formulations at acid pH, particularly below pH 5. Thus it is preferred to use longer-chain polyphosphates, in particular Glass H with an average chain length of about 21. It is believed such longer-chain polyphosphates when undergoing hydrolysis produce shorter-chain polyphosphates which are still effective to deposit onto teeth and provide a stain preventive benefit.

Other polyphosphorylated compounds may be used in addition to or instead of the polyphosphate, in particular polyphosphorylated inositol compounds such as phytic acid, myo-inositol pentakis(dihydrogen phosphate); myo-inositol tetrakis(dihydrogen phosphate), myo-inositol trikis(dihydrogen phosphate), and an alkali metal, alkaline earth metal or ammonium salt thereof Preferred herein is phytic acid, also known as myo-inositol 1,2,3,4,5,6-hexakis (dihydrogen phosphate) or inositol hexaphosphoric acid, and its alkali metal, alkaline earth metal or ammonium salts. Herein, the term “phytate” includes phytic acid and its salts as well as the other polyphosphorylated inositol compounds.

The amount of tooth substantive agent will typically be from about 0.1% to about 35% by weight of the total oral composition. In dentifrice formulations, the amount is preferably from about 2% to about 30%, more preferably from about 5% to about 25%, and most preferably from about 6% to about 20%. In mouthrinse compositions, the amount of tooth substantive agent is preferably from about 0.1% to 5% and more preferably from about 0.5% to about 3%.

In addition to creating the surface modifying effects, the tooth substantive agent may also function to solubilize insoluble salts. For example, Glass H has been found to solubilize insoluble stannous salts. Thus, in compositions containing stannous fluoride for example, Glass H contributes to decreasing the stain promoting effect of stannous.

### Chelating Agents

Another optional agent is a chelating agent, also called sequestrants, such as gluconic acid, tartaric acid, citric acid and pharmaceutically-acceptable salts thereof. Chelating agents are able to complex calcium found in the cell walls of the bacteria. Chelating agents can also disrupt plaque by removing calcium from the calcium bridges which help hold this biomass intact. However, it is not desired to use a chelating agent which has an affinity for calcium that is too high, as this may result in tooth demineralization, which is contrary to the objects and intentions of the present invention. Suitable chelating agents will generally have a calcium binding constant of about 10¹ to 10⁵ to provide improved cleaning with reduced plaque and calculus formation. Chelating agents also have the ability to complex with metallic ions and thus aid in preventing their adverse effects on the stability or appearance of products. Chelation of ions, such as iron or copper, helps retard oxidative deterioration of finished products.

Examples of suitable chelating agents are sodium or potassium gluconate and citrate; citric acid/alkali metal citrate combination; disodium tartrate; dipotassium tartrate; sodium potassium tartrate; sodium hydrogen tartrate; potassium hydrogen tartrate; sodium, potassium or ammonium polyphosphates and mixtures thereof The chelating agent may be used from about 0.1% to about 2.5%, preferably from about 0.5% to about 2.5% and more preferably from about 1.0% to about 2.5%.

Still other chelating agents suitable for use in the present invention are the anionic polymeric polycarboxylates. Such materials are well known in the art, being employed in the form of their free acids or partially or preferably fully neutralized water soluble alkali metal (e.g. potassium and preferably sodium) or ammonium salts. Examples are 1:4 to 4:1 copolymers of maleic anhydride or acid with another polymerizable ethylenically unsaturated monomer, preferably methyl vinyl ether (methoxyethylene) having a molecular weight (M.W.) of about 30,000 to about 1,000,000. These copolymers are available for example as Gantrez AN 139 (M.W. 500,000), AN 119 (M.W. 250,000) and S-97 Pharmaceutical Grade (M.W. 70,000), of GAF Chemicals Corporation.

Other operative polymeric polycarboxylates include the 1:1 copolymers of maleic anhydride with ethyl acrylate, hydroxyethyl methacrylate, N-vinyl-2-pyrrolidone, or ethylene, the latter being available for example as Monsanto EMA No. 1103, M.W. 10,000 and EMA Grade 61, and 1:1 copolymers of acrylic acid with methyl or hydroxyethyl methacrylate, methyl or ethyl acrylate, isobutyl vinyl ether or N-vinyl-2-pyrrolidone.

Additional operative polymeric polycarboxylates are disclosed in U.S. Pat. No. 4,138,477 to Gaffar and U.S. Pat. No. 4,183,914 to Gaffar et al. and include copolymers of maleic anhydride with styrene, isobutylene or ethyl vinyl ether; polyacrylic, polyitaconic and polymaleic acids; and sulfoacrylic oligomers of M.W. as low as 1,000 available as Uniroyal ND-2.

### Surfactants

The present compositions may also comprise surfactants, also commonly referred to as sudsing agents. Suitable surfactants are those which are reasonably stable and foam throughout a wide pH range. The surfactant may be anionic, nonionic, amphoteric, zwitterionic, cationic, or mixtures thereof.

Anionic surfactants useful herein include the water-soluble salts of alkyl sulfates having from 8 to 20 carbon atoms in the alkyl radical (e.g., sodium alkyl sulfate) and the water-soluble salts of sulfonated monoglycerides of fatty acids having from 8 to 20 carbon atoms. Sodium lauryl sulfate (SLS) and sodium coconut monoglyceride sulfonates are examples of anionic surfactants of this type. Other suitable anionic surfactants are sarcosinates, such as sodium lauroyl sarcosinate, taurates, sodium lauryl sulfoacetate, sodium lauroyl isethionate, sodium laureth carboxylate, and sodium dodecyl benzenesulfonate. Mixtures of anionic surfactants can also be employed. Many suitable anionic surfactants are disclosed by Agricola et al., U.S. Pat. No. 3,959,458. The present composition typically comprises an anionic surfactant at a level of from about 0.025% to about 9%, from about 0.05% to about 5% or from about 0.1% to about 1%.

Another suitable surfactant is one selected from the group consisting of sarcosinate surfactants, isethionate surfactants and taurate surfactants. Preferred for use herein are alkali metal or ammonium salts of these surfactants, such as the sodium and potassium salts of the following: lauroyl sarcosinate, myristoyl sarcosinate, palmitoyl sarcosinate, stearoyl sarcosinate and oleoyl sarcosinate. The sarcosinate surfactant may be present in the present compositions from about 0.1% to about 2.5%, preferably from about 0.5% to about 2.0% by weight.

Cationic surfactants useful in the present invention include derivatives of quaternary ammonium compounds having one long alkyl chain containing from about 8 to 18 carbon atoms such as lauryl trimethylammonium chloride; cetyl pyridinium chloride; cetyl trimethylammonium bromide; coconut alkyltrimethylammonium nitrite; cetyl pyridinium fluoride; etc. Preferred compounds are the quaternary ammonium fluorides described in U.S. Pat. No. 3,535,421 to Briner et al., where said quaternary ammonium fluorides have detergent properties. Certain cationic surfactants can also act as germicides in the compositions disclosed herein. Cationic surfactants such as chlorhexidine, although suitable for use in the current invention, are not preferred due to their capacity to stain the oral cavity's hard tissues. Persons skilled in the art are aware of this possibility and should incorporate cationic surfactants with this limitation in mind.

Nonionic surfactants that can be used in the compositions of the present invention include compounds produced by the condensation of alkylene oxide groups (hydrophilic in nature) with an organic hydrophobic compound which may be aliphatic or alkylaromatic in nature. Examples of suitable nonionic surfactants include the Pluronics, polyethylene oxide condensates of alkyl phenols, products derived from the condensation of ethylene oxide with the reaction product of propylene oxide and ethylene diamine, ethylene oxide condensates of aliphatic alcohols, long chain tertiary amine oxides, long chain tertiary phosphine oxides, long chain dialkyl sulfoxides and mixtures of such materials.

Zwitterionic synthetic surfactants useful in the present invention include derivatives of aliphatic quaternary ammonium, phosphonium, and sulfonium compounds, in which the aliphatic radicals can be straight chain or branched, and wherein one of the aliphatic substituents contains from about 8 to 18 carbon atoms and one contains an anionic water-solubilizing group, e.g., carboxy, sulfonate, sulfate, phosphate or phosphonate.

Suitable betaine surfactants are disclosed in U.S. Pat. No. 5,180,577 to Polefka et al. Typical alkyl dimethyl betaines include decyl betaine or 2-(N-decyl-N,N-dimethylammonio)acetate, coco betaine or 2-(N-coco-N,N-dimethyl ammonio)acetate, myristyl betaine, palmityl betaine, lauryl betaine, cetyl betaine, cetyl betaine, stearyl betaine, etc. The amidobetaines are exemplified by cocoamidoethyl betaine, cocoamidopropyl betaine, and lauramidopropyl betaine.

### Thickening Agents

In preparing toothpaste or gels, thickening agents are added to provide a desirable consistency to the composition, to provide desirable active release characteristics upon use, to provide shelf stability, and to provide stability of the composition, etc. Suitable thickening agents include one or a combination of carboxyvinyl polymers, carrageenan, hydroxyethyl cellulose (HEC), natural and synthetic clays (e.g., Veegum and laponite) and water soluble salts of cellulose ethers such as sodium carboxymethylcellulose (CMC) and sodium carboxymethyl hydroxyethyl cellulose. Natural gums such as gum karaya, xanthan gum, gum arabic, and gum tragacanth can also be used. Colloidal magnesium aluminum silicate or finely divided silica can be used as part of the thickening agent to further improve texture.

Suitable carboxyvinyl polymers useful as thickening or gelling agents include carbomers which are homopolymers of acrylic acid crosslinked with an alkyl ether of pentaerythritol or an alkyl ether of sucrose. Carbomers are commercially available from B.F. Goodrich as the Carbopol® series, including Carbopol 934, 940, 941, 956, and mixtures thereof.

Thickening agents are typically present in an amount from about 0.1% to about 15%, preferably from about 2% to about 10%, more preferably from about 4% to about 8%, by weight of the total toothpaste or gel composition, can be used. Higher concentrations may be used for chewing gums, lozenges and breath mints, sachets, non-abrasive gels and subgingival gels.

### Humectants

Another optional carrier material of the present compositions is a humectant. The humectant serves to keep toothpaste compositions from hardening upon exposure to air, to give compositions a moist feel to the mouth, and, for particular humectants, to impart desirable sweetness of flavor to toothpaste compositions. The humectant, on a pure humectant basis, generally comprises from about 0% to about 70%, preferably from about 5% to about 25%, by weight of the compositions herein. Suitable humectants for use in compositions of the subject invention include edible polyhydric alcohols such as glycerin, sorbitol, xylitol, butylene glycol, polyethylene glycol, propylene glycol and trimethyl glycine.

### Miscellaneous Carrier Materials

Water employed in the preparation of commercially suitable oral compositions should preferably be of low ion content and free of organic impurities. Water may comprise up to about 99% by weight of the aqueous compositions herein. These amounts of water include the free water which is added plus that which is introduced with other materials, such as with sorbitol.

The present invention may also include an alkali metal bicarbonate salt, which may serve a number of functions including abrasive, deodorant, buffering and adjusting pH. Alkali metal bicarbonate salts are soluble in water and unless stabilized, tend to release carbon dioxide in an aqueous system. Sodium bicarbonate, also known as baking soda, is a commonly used alkali metal bicarbonate salt. The present composition may contain from about 0.5% to about 30% by weight of an alkali metal bicarbonate salt.

The pH of the present compositions may be adjusted through the use of buffering agents. Buffering agents, as used herein, refer to agents that can be used to adjust the pH of aqueous compositions such as mouthrinses and dental solutions preferably to a range of about pH 4.0 to about pH 8.0. Buffering agents include sodium bicarbonate, monosodium phosphate, trisodium phosphate, sodium hydroxide, sodium carbonate, sodium acid pyrophosphate, citric acid, and sodium citrate and are typically included at a level of from about 0.5% to about 10% by weight.

Poloxamers may be employed in the present compositions. A poloxamer is classified as a nonionic surfactant and may also function as an emulsifying agent, binder, stabilizer, and other related functions. Poloxamers are difunctional block-polymers terminating in primary hydroxyl groups with molecular weights ranging from 1,000 to above 15,000. Poloxamers are sold under the tradename of Pluronics and Pluraflo by BASF including Poloxamer 407 and Pluraflo L4370.

Other emulsifying agents that may be used include polymeric emulsifiers such as the Pemulen® series available from B.F. Goodrich, and which are predominantly high molecular weight polyacrylic acid polymers useful as emulsifiers for hydrophobic substances.

Titanium dioxide may also be added to the present compositions as coloring or opacifying agent typically at a level of from about 0.25% to about 5% by weight.

Other optional agents that may be used in the present compositions include dimethicone copolyols selected from alkyl- and alkoxy-dimethicone copolyols, such as C12 to C20 alkyl dimethicone copolyols and mixtures thereof, as aid in providing positive tooth feel benefits. Highly preferred is cetyl dimethicone copolyol marketed under the trade name Abil EM90. The dimethicone copolyol is generally present from about 0.01% to about 25%, preferably from about 0.1% to about 5%, more preferably from about 0.5% to about 1.5% by weight.

### Respiratory Ingredients

The personal care compositions for nasal and throat care can comprise a wide range of respiratory ingredients. Nonlimiting examples include analgesics, anticholinergics, antihistamines, anti-inflammatories, antipyretics, antitussives, antivirals, decongestants, expectorants, mucolytics, and combinations thereof.

Example of decongestants include: oxymetazoline, phenylephrine, xylometazoline, naphazoline, 1-desoxyephedrine, ephedrine, propylhexedrine, pseudoephedrine, and phenylpropanolamine. Example of anticholinergics include: ipratropium, chlorpheniramine, brompheniramine, diphenhydramine, doxylamine, clemastine, and triprolidine. Common analgesics, anti-inflammatories and antipyretics include: ibuprofen, ketoprofen, diclofenac, naproxen, acetaminophen, and aspirin. Example of antivirals include: amantidine, rimantidine, pleconaril, zanamivir, and oseltamivir. Examples of antitussives include codeine, dextromethorphan, chlophedianol and levodropropizine. Examples of expectorants include guaifenesin. Examples of mucolytics include ambroxol and N-acetylcysteine. Examples of antihistamines include diphenhydramine, doxylamine, triprolidine, clemastine, pheniramine, chlorpheniramine, brompheniramine, Dexbrompheniramine, loratadine, cetirizine and fexofenadine, Amlexanox, Alkylamine Derivatives, Cromolyn, Acrivastine, Ibudilast, Bamipine, Ketotifen, Nedocromil, Omalizumab, Dimethindene, Oxatomide, Pemirolast, Pyrrobutamine, Pentigetide, Thenaldine, Picumast, Tolpropamine, Ramatroban, Triprolidine, Repirinast, Suplatast Tosylate Aminoalkylethers, Tazanolast, Bromodiphenhydramine, Tranilast, Carbinoxamine, Traxanox, Chlorphenoxamine, Diphenhydramine, Diphenylpyaline, Doxylamine, Embramine, p-Methyldiphenhydramine, Moxastine, Orphenadrine, Phenyltoloxamine, Setastine, Ethylenediamine Derivatives, Chloropyramine, Chlorothen, Methapyrilene, Pyrilamine, Talastine, Thenyldiamine, Thonzylamine Hydrochloride, Tripelennamine, Piperazines, Chlorcyclizine, Clocinizine, Homochlorcyclizine, Hydroxyzine, Tricyclics, Phenothiazines, Mequitazine, Promethazine, Thiazinamium Methylsulfate, Other Tricyclics, Azatadine, Cyproheptadine, Deptropine, Desloratadine, Isothipendyl, Olopatadine, Rupatadine, Antazoline, Astemizole, Azelastine, Bepotastine, Clemizole, Ebastine, Emedastine, Epinastine, Levocabastine, Mebhydroline, Mizolastine, Phenindamine, Terfenadine, Tritoqualine.

The composition may comprise an amount of respiratory ingredient in the range of from about 0% to about 15%, alternatively 0.0001% to about 10%, alternatively from about 0.001% to about 7%, and alternatively from about 0.01% to about 5%, all by weight of the composition.

### Method of Use

The present invention also relates to methods for controlling bacterial activity in the oral which cause undesirable conditions including plaque, caries, calculus, gingivitis, periodontal disease and malodor. The benefits of these compositions may increase over time when the composition is used repeatedly.

The method of use or treatment herein may comprise contacting a subject's dental enamel surfaces and mucosa in the mouth with the oral compositions according to the present invention. The method may comprise brushing with a dentifrice or rinsing with a dentifrice slurry or mouthrinse. Other methods include contacting the topical oral gel, denture product, mouthspray, or other form with the subject's teeth and oral mucosa. The subject may be any person or animal whose tooth surface is contacted with the oral composition. By animal is meant to include household pets or other domestic animals, or animals kept in captivity.

For example, a method of treatment may include a person brushing a dog's teeth with one of the dentifrice compositions. Another example would include rinsing a cat's mouth with an oral composition for a sufficient amount of time to see a benefit. Pet care products such as chews and toys may be formulated to contain the present oral compositions. The composition may be incorporated into a relatively supple but strong and durable material such as rawhide, ropes made from natural or synthetic fibers, and polymeric articles made from nylon, polyester or thermoplastic polyurethane. As the animal chews, licks or gnaws the product, the incorporated active elements are released into the animal's oral cavity into a salivary medium, comparable to an effective brushing or rinsing.

Other methods of use include cleansing and disinfecting hands and skin using sanitizing compositions or wipes containing the present antimicrobial blend of essential oil materials. Or a throat spray containing the present blend may be used to treat a throat infection.

When the composition is a respiratory composition the term “orally administering” and/or “administering” with respect to the human/mammal means that the human/mammal ingests or is directed to ingest, or does ingest, or deliver, or chew, or drink, or spray, or place in mouth, one or more of the present respiratory composition. The human/mammal may be directed to deliver the respiratory composition to the site that the human/mammal intends to treat for example the mouth and/or throat. The human/mammal may be directed to ingest or deliver or chew, or drink, or spray, or place in mouth the composition, such direction and or deliver may be that which instructs and/or informs the human that use of the composition may and/or will provide relief from the respiratory symptom (e.g., symptomatic relief, whether temporary or permanent) for example, relief from coughing and/or sore throat. The relief can be instant or on demand. For example, such direction may be oral direction (e.g., through oral instruction from, for example, a physician, pharmacist, or other health professional), radio or television media (e.g., advertisement), or written direction (e.g., through written direction from, for example, a physician, pharmacist, or other health professional (e.g., scripts), sales professional organization (e.g., through, for example, marketing brochures, pamphlets, or other instructive paraphernalia), written media (e.g., internet, electronic mail, or other computer-related media)), and/or packaging associated with the composition (e.g., a label present on a delivery device holding the preparation). As used herein, “written” means through words, pictures, symbols, and/or other visible or tactile descriptors. Such information need not utilize the actual words used herein, for example, “respiratory”, “symptom”, or “mammal”, but rather use of words, pictures, symbols, tactile means, and the like conveying the same or similar meaning are contemplated within the scope of this invention.

In a further embodiment, the respiratory composition is directed to methods of treating and providing cough relief on demand comprising administering a preparation as described herein to a mammal in need of such treatment. As further used herein, “treatment” and/or “providing relief”, with respect to cough relief, mean that administration of the referenced respiratory preparation prevents, alleviates, ameliorates, inhibits, or mitigates one or more symptoms of the condition.

The present invention can also be directed to methods of “prevention” including preventing a cough or its associated symptoms from occurring in a mammal, for example when the mammal is predisposed to acquiring the symptoms of coughing, inhibiting the onset of coughing or its associated symptoms; and/or alleviating, reversing, or curing the coughing episode or its associated symptoms.

Administration may be on an as-needed or as-desired basis, for example, once-monthly, once-weekly, or daily, including multiple times daily, for example, at least once daily, from one to about six times daily, from about two to about four times daily, or about three times daily. The amount of respiratory composition administered may be dependent on a variety of factors, including the general quality of health of the mammal, age, gender, weight, or severity of symptoms.

### EXAMPLES

The following examples further describe and demonstrate embodiments within the scope of the present invention. These examples are given solely for the purpose of illustration and are not to be construed as limitations of the present invention as many variations thereof are possible without departing from the spirit and scope.

### Example I

### Dentifrice Compositions

Dentifrice compositions according to the present invention Ia-Ik are shown below with amounts of ingredients in weight %. These compositions are made using conventional methods. In consumer sensory tests, compositions according to the present invention were rated as having a pleasant, long-lasting, natural, light herbal taste and providing cleaning and freshening of the mouth without the typical burn and aftertaste.

| Ingredient | Ia | Ib | Ic | Id | Ie | If |
|---|---|---|---|---|---|---|
| Peppermint Flavor | 1.00 | | | | | |
| Spearmint Flavor | | 1.00 | | | 1.00 | 1.00 |
| Wintergreen Flavor | | | 1.00 | | | |
| Cinnamon Flavor | | | | 1.00 | | |
| Carvacrol | | 0.20 | | 0.20 | 0.06 | 0.04 |
| Eucalyptol | | 0.10 | 0.10 | 0.20 | 0.30 | 0.19 |
| Eugenol | 0.10 | | 0.25 | 0.20 | 0.18 | 0.11 |
| Geraniol | | 0.30 | 0.15 | 0.20 | 0.24 | 0.15 |
| Citral | 0.40 | | 0.25 | 0.20 | 0.02 | 0.01 |
| Sorbitol 70% Solution | 65.0 | 65.0 | 65.0 | 65.0 | 65.0 | 65.0 |
| Sodium Lauryl Sulfate | 4.00 | 4.00 | 4.00 | 4.00 | 4.00 | 4.00 |
| 28% Soln | | | | | | |
| Na Saccharin | 0.40 | 0.40 | 0.40 | 0.40 | 0.40 | 0.40 |
| Silica Abrasive | 20.0 | 20.0 | 20.0 | 20.0 | 20.0 | 20.0 |
| Na Hydroxide 50% Solution | 0.70 | 0.70 | 0.70 | 0.70 | 0.70 | 0.70 |
| Na Acid Pyrophosphate | 1.30 | 1.30 | 1.30 | 1.30 | 1.30 | 1.30 |
| Xanthan Gum | 0.20 | 0.20 | 0.20 | 0.20 | 0.20 | 0.20 |
| Carbomer 956 | 0.40 | 0.40 | 0.40 | 0.40 | 0.40 | 0.40 |
| Na Carboxymethylcellulose | 0.20 | 0.20 | 0.20 | 0.20 | 0.20 | 0.20 |
| Water | QS | QS | QS | QS | QS | QS |

| Ingredient | Ig | Ih | Ii | Ij | Ik |
|---|---|---|---|---|---|
| Peppermint Flavor | 0.50 | | 0.70 | 0.30 | 0.70 |
| Spearmint Flavor | 0.50 | | 0.30 | 0.70 | |
| Wintergreen Flavor | | 0.70 | | | |
| Cinnamon Flavor | | 0.30 | | | 0.30 |
| Eucalyptol | | 0.175 | 0.35 | 0.35 | |
| Eugenol | 0.50 | 0.25 | | | 0.35 |
| Geraniol | | 0.325 | 0.65 | | 0.35 |
| Citral | 0.50 | 0.25 | | 0.35 | |
| Zinc Chloride | | | 1.00 | | |
| Zinc Citrate | | 2.00 | | | |
| Zinc Lactate | 2.00 | | | 1.00 | 2.00 |
| Sorbitol 70% Solution | 65.0 | 65.0 | 65.0 | 65.0 | 65.0 |
| Sodium Lauryl Sulfate 28% Soln | 4.00 | 4.00 | 4.00 | 4.00 | 4.00 |
| Na Saccharin | 0.40 | 0.40 | 0.40 | 0.40 | 0.40 |
| Silica Abrasive | 20.0 | 20.0 | 20.0 | 20.0 | 20.0 |
| Na Hydroxide 50% Solution | 0.70 | 0.70 | 0.70 | 0.70 | 0.70 |
| Na Acid Pyrophosphate | 1.30 | 1.30 | 1.30 | 1.30 | 1.30 |
| Xanthan Gum | 0.20 | 0.20 | 0.20 | 0.20 | 0.20 |
| Carbomer 956 | 0.40 | 0.40 | 0.40 | 0.40 | 0.40 |
| Na Carboxymethylcellulose | 0.20 | 0.20 | 0.20 | 0.20 | 0.20 |
| Water | QS | QS | QS | QS | QS |

### Example II

### Mouthrinse Compositions

Mouthrinse compositions according to the present invention (IIa-IIj) are shown below with amounts of ingredients in weight %. These compositions are made using conventional methods.

| Ingredient | IIa | IIb | IIc | IId |
|---|---|---|---|---|
| Peppermint Flavor | 0.10 | | | |
| Citrus Flavor | | 0.10 | | |
| Wintergreen Flavor | | | 0.10 | |
| Cinnamon Flavor | | | | 0.10 |
| Carvacrol | | | 0.03 | 0.02 |
| Eucalyptol | 0.04 | | | 0.02 |
| Eugenol | 0.02 | 0.01 | 0.01 | 0.02 |
| Geraniol | 0.015 | 0.01 | 0.02 | |
| Citral | 0.05 | 0.04 | 0.025 | 0.02 |
| Glycerin | 20.0 | 20.0 | 20.0 | 20.0 |
| Ethanol | 5.0 | 10.0 | | 20.0 |
| Poloxamer 407 | 1.00 | 0.50 | 1.00 | 0.50 |
| Na Saccharin | 0.05 | 0.03 | 0.03 | 0.05 |
| Cetylpyridinium Chloride | | 0.07 | | 0.07 |
| Water | QS | QS | QS | QS |

| Ingredient | IIe | IIf | IIg | IIh | IIi | IIj |
|---|---|---|---|---|---|---|
| Peppermint | | | 0.07 | 0.07 | 0.50 | |
| Flavor | | | | | | |
| Citrus Flavor | | | | 0.03 | 0.50 | 0.10 |
| Wintergreen | 0.10 | 0.07 | | | | |
| Flavor | | | | | | |
| Cinnamon Flavor | | 0.05 | 0.03 | | | |
| Eucalyptol | | 0.0263 | 0.07 | 0.075 | | |
| Eugenol | 0.075 | 0.0375 | | | 0.075 | 0.075 |
| Geraniol | | 0.0487 | 0.14 | | | 0.075 |
| Citral | 0.075 | 0.0375 | | 0.075 | 0.075 | |
| Glycerin | 20.0 | 20.0 | 20.0 | 20.0 | | |
| Zinc Chloride | | | 0.10 | | | |
| Zinc Citrate | | 0.20 | | | | 0.20 |
| Zinc Lactate | 0.20 | | | 0.10 | 0.20 | |
| Ethanol | 5.0 | 10.0 | | 20.0 | 20.0 | 15.0 |
| Poloxamer 407 | 1.00 | 0.50 | 1.00 | 0.50 | 0.50 | 0.50 |
| Na Saccharin | 0.05 | 0.03 | 0.03 | 0.05 | 0.05 | 0.05 |
| Cetylpyridinium | | 0.07 | | 0.07 | 0.07 | 0.07 |
| Chloride | | | | | | |
| Water | QS | QS | QS | QS | QS | QS |

### Example III

### Hand Sanitizer Compositions

Hand sanitizer compositions (IIIa-IIIh) containing the present antimicrobial blends are shown below with amounts of ingredients in weight %. These compositions are made using conventional methods.

| Ingredient | IIIa | IIIb | IIIc | IIId |
|---|---|---|---|---|
| Carvacrol | 0.20 | 0.06 | 0.04 | |
| Eucalyptol | 0.20 | 0.30 | 0.19 | |
| Eugenol | 0.20 | 0.18 | 0.11 | 0.50 |
| Geraniol | 0.20 | 0.24 | 0.15 | |
| Citral | 0.20 | 0.02 | 0.01 | 0.50 |
| Ethanol | 15.0 | 15.0 | 15.0 | 15.0 |
| L-Pyrrolidone Carboxylic Acid | 4.20 | 4.20 | 4.20 | 4.20 |
| Succinic Acid | 2.29 | 2.29 | 2.29 | 2.29 |
| Disodium Succinate Hexahydrate | 0.71 | 0.71 | 0.71 | 0.71 |
| Veragel¹ | 1.00 | 1.00 | 1.00 | 1.00 |
| Cocamidopropyl Hydroxysultaine | 0.50 | 0.50 | 0.50 | 0.50 |
| Ammonium Lauryl Sulfate | 0.90 | 0.90 | 0.90 | 0.90 |
| Sodium Olefin Sulfonate | 0.50 | 0.50 | 0.50 | 0.50 |
| Plexajel² | 1.00 | 1.00 | 1.00 | 1.00 |
| Water | QS | QS | QS | QS |

¹Aloe Vera gel (Aloe Barbadensis extract)
²Plexajel ASC supplied by Guardian Laboratories is a mixture of water, glycerin, Polyquaternium-4 and polyacrylamidomethylpropane sulfonic acid.

| | | | | |
|---|---|---|---|---|
| Ingredient | IIIe | IIf | IIIg | IIIh |

| | | | | |
|---|---|---|---|---|
| Eucalyptol | 0.175 | 0.35 | 0.35 | |
| Eugenol | 0.25 | | | 0.35 |
| Geraniol | 0.325 | 0.65 | | 0.35 |
| Citral | 0.25 | | 0.35 | |
| Ethanol | 15.0 | 15.0 | 15.0 | 15.0 |
| L-Pyrrolidone Carboxylic Acid | 4.20 | 4.20 | 4.20 | 4.20 |
| Succinic Acid | 2.29 | 2.29 | 2.29 | 2.29 |
| Disodium Succinate Hexahydrate | 0.71 | 0.71 | 0.71 | 0.71 |
| Veragel¹ | 1.00 | 1.00 | 1.00 | 1.00 |
| Cocamidopropyl Hydroxysultaine | 0.50 | 0.50 | 0.50 | 0.50 |
| Ammonium Lauryl Sulfate | 0.90 | 0.90 | 0.90 | 0.90 |
| Sodium Olefin Sulfonate | 0.50 | 0.50 | 0.50 | 0.50 |
| Plexajel² | 1.00 | 1.00 | 1.00 | 1.00 |
| Water | QS | QS | QS | QS |

¹Aloe Vera gel (Aloe Barbadensis extract)
²Plexajel ASC supplied by Guardian Laboratories is a mixture of water, glycerin, Polyquaternium-4 and polyacrylamidomethylpropane sulfonic acid.

### Example IV

### Respiratory Compositions

Respiratory compositions are shown below with amounts of ingredients in weight %. Examples #1-#8 are liquid compositions made using conventional methods and may be used for example as a throat spray or gargle.

| Ingredient | #1 | #2 | #3 | #4 |
|---|---|---|---|---|
| Carvacrol | 0.25 | 0.06 | 0.04 | |
| Eucalyptol | 0.25 | 0.30 | 0.19 | |
| Eugenol | 0.25 | 0.18 | 0.11 | 0.15 |
| Geraniol | 0.25 | 0.24 | 0.15 | |
| Citral | 0.25 | 0.02 | 0.01 | 0.15 |
| Polyoxyl 40 Stearate | 0.75 | 0.75 | 0.75 | 0.75 |
| Polyethylene Oxide | | 0.25 | | 0.25 |
| Sodium Carboxymethylcellulose | 0.42 | 0.45 | 0.42 | 0.45 |
| Flavor | 0.50 | 1.00 | 0.30 | 0.30 |
| Na Saccharin | 0.50 | 0.30 | | 0.20 |
| Sucralose | | 0.10 | 0.20 | |
| Sodium Benzoate | 0.10 | 0.10 | 0.10 | 0.10 |
| Benzoic Acid | 0.13 | 0.13 | 0.13 | 0.13 |
| Propylene Glycol | 15.0 | 8.0 | 15.0 | 8.0 |
| Sorbitol Solution | 15.0 | 15.0 | 15.0 | 15.0 |
| Water | QS | QS | QS | QS |

| Ingredient | #5 | #6 | #7 | #8 |
|---|---|---|---|---|
| Eucalyptol | 0.175 | 0.35 | 0.35 | |
| Eugenol | 0.25 | | | 0.50 |
| Geraniol | 0.325 | 0.65 | | 0.50 |
| Citral | 0.25 | | 0.35 | |
| Polyoxyl 40 Stearate | 0.75 | 0.75 | 0.75 | 0.75 |
| Polyethylene Oxide | | 0.25 | | 0.25 |
| Sodium Carboxymethylcellulose | 0.42 | 0.45 | 0.42 | 0.45 |
| Flavor | 0.50 | 0.50 | 0.30 | 0.50 |
| Na Saccharin | 0.40 | 0.50 | 0.40 | 0.30 |
| Sucralose | 0.10 | | | 0.10 |
| Sodium Benzoate | 0.10 | 0.10 | 0.10 | 0.10 |
| Benzoic Acid | 0.13 | 0.13 | 0.13 | 0.13 |
| Propylene Glycol | 15.0 | 8.0 | 15.0 | 8.0 |
| Sorbitol Solution | 15.0 | 15.0 | 15.0 | 15.0 |
| Water | QS | QS | QS | QS |

Examples #9-#16 can be made by first adding water, citric acid, sodium CMC, polyoxyl 40 stearate, and or polyethylene oxide to a clean vessel. The contents are stirred until the CMC disperses. In a second separate vessel propylene glycol, glycerin, sucrose, sucralose, flavors and flavoring agents, salivation agent and sodium benzoate are added and stirred until dissolved. The two mixtures are then combined and mixed until homogenous and then placed in a delivery device comprising the material PET.

| Ingredient | #9 | #10 | #11 | #12 | # 13 | # 14 | # 15 | # 16 |
|---|---|---|---|---|---|---|---|---|
| Peppermint | 1.0 | | | | | 2.0 | | |
| Flavor | | | | | | | | |
| Spearmint | | 1.0 | | | 3.0 | | 1.0 | 1.0 |
| Flavor | | | | | | | | |
| Wintergreen | | | 1.0 | | | | | |
| Flavor | | | | | | | | |
| Cinnamon | | | | 1.0 | | | | |
| Flavor | | | | | | | | |
| Carvacrol | | 0.20 | | 0.20 | 0.06 | 0.04 | 0.20 | 0.20 |
| Eucalyptol | | 0.10 | 0.10 | 0.20 | 0.30 | 0.19 | 0.10 | 0.10 |
| Eugenol | 0.10 | | 0.25 | 0.20 | 0.18 | 0.11 | | |
| Geraniol | | 0.30 | 0.15 | 0.20 | 0.24 | 0.15 | 0.30 | 0.30 |
| Citral | 0.40 | | 0.25 | 0.20 | 0.02 | 0.01 | | |
| Propylene | 40.0 | 15.0 | 15.0 | 15.0 | 15.0 | 15.0 | 40.0 | 40.0 |
| Glycol | | | | | | | | |
| Sodium CMC | 0.45 | 0.5 | 0.5 | 0.5 | 0.5 | 0.5 | 0.45 | 0.45 |
| Citric Acid | 0.5 | 0.4 | 0.4 | 0.4 | 0.4 | 0.4 | 0.5 | 0.5 |
| Sucrose | 14 | 20.0 | 20.0 | 20.0 | 20.0 | 20.0 | 14.0 | 14.0 |
| Sucralose | 0.05 | 0.08 | 0.08 | 0.08 | 0.08 | 0.08 | 0.05 | 0.05 |
| Glycerin | 10.0 | 1.3 | 1.3 | 1.3 | 1.3 | 1.3 | 10.0 | 10.0 |
| Sorbitol 70% | | 15.0 | 15.0 | 15.0 | 15.0 | 15.0 | | |
| Solution | | | | | | | | |
| Polyoxyl | | 0.6 | 0.6 | 0.6 | | 0.6 | | |
| 40 Stearate | | | | | | | | |
| Polyethylene | | 0.2 | 0.2 | 0.2 | | 0.2 | | |
| Oxide | | | | | | | | |
| Sodium | 0.01 | 0.01 | 0.01 | 0.01 | 0.01 | 0.01 | 0.01 | 0.01 |
| Benzoate | | | | | | | | |
| Salivation | | | | | | | 0.02 | 0.10 |
| Agent¹ | | | | | | | | |
| Water | QS | QS | QS | QS | QS | QS | QS | QS |

¹Optaflow ® supplied by Symrise is an example of a salivation agent that may be used.

Examples #17-#16 can be made by first adding water, citric acid, sodium CMC and poloxamer 407 to a clean vessel. The contents are stirred until the ingredients disperse. In a separate vessel the xanthan gum, guar gum and glycerine are mixed until the gums dissolve and disperse. In a third separate clean vessel the propylene glycol, sucrose, sucralose, flavors, sodium citrate and sodium benzoate are added and stirred until dissolved. The three mixtures are then combined and mixed until homogenous and then placed in a delivery device comprising the material PET.

Examples #21-#24 can be made by first adding water, citric acid, and sodium CMC to a clean vessel. The contents are stirred until the CMC disperses. In a separate clean vessel the high fructose corn syrup, propylene glycol, respiratory ingredients (Chlorpheniramine Maleate, Guaifenesin, Dextromethorphan HBr) glycerin, menthol, sucrose, sucralose, flavors, sodium citrate and sodium benzoate are added and stirred until dissolved. The two mixtures are then combined and mixed until homogenous and then placed in a delivery device comprising the material PET.

| Ingredient | #17 | #18 | #19 | #20 |
|---|---|---|---|---|
| Citric Acid | 0.3 | 0.3 | 0.3 | 0.3 |
| Sodium CMC | 0.3 | 0.3 | 0.3 | 0.30 |
| Propylene Glycol | 10.0 | 10.0 | 10.0 | 40.0 |
| Glycerin | | | 10.0 | 20.0 |
| Sucrose | 14.0 | 14.0 | 14.0 | 14.0 |
| Sodium Saccharin | 0.14 | 0.14 | 0.14 | 0.14 |
| Sodium Benzoate | 0.01 | 0.01 | 0.01 | 0.01 |
| Sodium Citrate Dihydrate | 0.45 | 0.45 | 0.45 | 0.45 |
| High Fructose Corn Syrup | 45.0 | 45.0 | 45.0 | 45.0 |
| Chlorpheniramine Maleate | 0.02 | | 0.02 | |
| Guaifenesin | 1.14 | 1.14 | 1.14 | |
| Dextromethorphan HBr | | | 0.67 | 0.67 |
| Peppermint Flavor | 0.10 | | | |
| Citrus Flavor | | 0.10 | | |
| Wintergreen Flavor | | | 0.10 | |
| Cinnamon Flavor | | | | 0.10 |
| Carvacrol | | | 0.03 | 0.02 |
| Eucalyptol | 0.04 | | | 0.02 |
| Eugenol | 0.02 | 0.01 | 0.01 | 0.02 |
| Geraniol | 0.015 | | 0.01 | 0.02 |
| Citral | 0.05 | 0.04 | 0.025 | 0.02 |
| USP Water | QS | QS | QS | QS |

| Ingredient | #21 | #22 | #23 | #24 |
|---|---|---|---|---|
| Peppermint Flavor | 0.10 | | | |
| Citrus Flavor | | 0.10 | | |
| Wintergreen Flavor | | | 0.10 | |
| Cinnamon Flavor | | | | 0.10 |
| Carvacrol | | | 0.03 | 0.02 |
| Eucalyptol | 0.04 | | | 0.02 |
| Eugenol | 0.02 | 0.01 | 0.01 | 0.02 |
| Geraniol | 0.015 | | 0.01 | 0.02 |
| Citral | 0.05 | 0.04 | 0.025 | 0.02 |
| Glycerin | 20.0 | 20.0 | 20.0 | 20.0 |
| Propylene Glycol | 40.0 | 40.0 | 25.0 | 10.0 |
| Sucrose | 14.0 | 14.0 | 14.0 | 14.0 |
| Sucralose | 0.05 | 0.05 | 0.05 | 0.05 |
| Sodium Benzoate | 0.01 | 0.01 | 0.01 | 0.07 |
| Citric Acid | 0.5 | 0.5 | 0.5 | 0.5 |
| Xanthan Gum | 0.65 | | | 0.55 |
| Poloxamer 407 | | | 0.55 | |
| Guar Gum | | 0.55 | | |
| USP Water | QS | QS | QS | QS |

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A blend of essential oil components comprising a first acyclic component selected from citral, neral, geranial, geraniol and nerol and a second cyclic-containing component selected from eucalyptol, eugenol and carvacrol and used in personal care compositions at a level of at least about 0.02% by weight to provide effective antimicrobial activity.

2. A blend according to claim 1 used in personal care compositions at a level of from about 0.02% to about 5.0% by weight.

3. A blend according to claim 1 prepared by blending individual or purified chemicals.

4. A blend according to claim 1 comprising at least two of said acyclic components and at least two of said cyclic-containing components.

5. A blend according to claim 1 comprising citral, geraniol, eucalyptol and eugenol, each component at a level of at least about 0.5% by weight of the blend.

6. A blend according to claim 1 comprising citral, geraniol, eucalyptol, eugenol and carvacrol, each component at a level of at least about 0.5% by weight of the blend.

7. Personal care products for use on skin, hair, oral cavity, nasal passages, throat and other mucosal surfaces comprising from about 0.02% to about 5% by weight of an antimicrobial blend of essential oil components, the blend comprising a first acyclic component selected from citral, neral, geranial, geraniol and nerol and a second cyclic-containing component selected from eucalyptol, eugenol and carvacrol.

8. An oral care composition comprising
(a) from at least about 0.02% by weight of the total composition of a blend of essential oil components as essential antimicrobial active, the blend comprising a first acyclic component 5 selected from citral, neral, geranial, geraniol and nerol and a second cyclic-containing component selected from eucalyptol, eugenol and carvacrol, and
(b) an orally-acceptable carrier,
wherein the composition provides effective antimicrobial activity against microorganisms involved in one or more undesirable oral cavity conditions selected from plaque, caries, calculus, gingivitis and breath malodor.

9. An oral care composition according to claim 8 comprising from about 0.05% to 5% by weight of the total composition of the antimicrobial blend.

10. An oral care composition according to claim 8 wherein the antimicrobial blend is prepared from individual or purified chemicals.

11. An oral care composition according to claim 8 further comprising an additional antimicrobial active.

12. An oral care composition according to claim 11 wherein the additional antimicrobial active is selected from cetylpyridinium chloride, zinc ion source, stannous ion source, copper ion source, a peroxide source, a chlorite source, chlorhexidine, triclosan, triclosan monophosphate and mixtures thereof.

13. An oral care composition according to claim 11 wherein the additional antimicrobial active is selected from o-cymen-5-ol, menthol, phenol, carvone, cinnamaldehyde, anethole, terpinene-4-ol, zingerone, allyl isothiocyanate, cuminaldehyde, hinokitiol, α-pinene, β-pinene, dipentene, benzyl alcohol, benzaldehyde, guaiacol, mixtures thereof and natural sources thereof.

14. An oral care composition according to claim 8 in a form selected from toothpaste, dentifrice, tooth gel, subgingival gel, mouthrinse, mousse, foam, denture product, mouthspray, lozenge, chewable tablet, and chewing gum.

15. A personal care composition according to claim 7 wherein said composition is a respiratory composition further comprising a respiratory ingredient.
